(19) United States
(12) Patent Application Publication
(10) Pub. No.: US 2010/0099747 A1
(43) Pub. Date: Apr. 22, 2010

(54) REDUCTION OF POLYALANINE-INDUCED PROTEIN AGGREGATES AND TOXICITY BY UBIQUILIN

(75) Inventors: Mervyn J. Monteiro, Columbia, MD (US); Hongmin Wang, Vermillion, SD (US)

Correspondence Address: INTELLECTUAL PROPERTY / TECHNOLOGY LAW, PO BOX 14329, RESEARCH TRIANGLE PARK, NC, 27709, US

(73) Assignee: UNIVERSITY OF MARYLAND BIOTECHNOLOGY INSTITUTE, Baltimore, MD (US)

(21) Appl. No.: 12/520,492

(22) PCT Filed: Dec. 21, 2007
(86) PCT No.: PCT/US07/88747

§ 371 Date: Nov. 12, 2009

Related U.S. Application Data
(60) Provisional application No. 60/876,451, filed on Dec. 21, 2006.

(51) Int. Cl. A61K 31/7088; C12N 5/071; A61P 25/28

(52) U.S. Cl. 514/44 R; 435/375

(57) ABSTRACT

A method for decreasing cell death in a cell exhibiting aggregation of polyalanine-containing proteins. The method includes introduction of an expression vector to a host cell comprising a nucleotide sequence encoding ubiquilin, followed by maintaining the transformed host cell under biological conditions sufficient for expression and accumulation of the ubiquilin in the host cell, wherein overexpression of ubiquilin reduces sensitivity of cell stress induced by expanded polyalanine proteins.

### CROSS-REFERENCE TO RELATED APPLICATION

This application claims the benefit under 35 USC 119 of U.S. Provisional Patent Application No. 60/876,451 filed on Dec. 21, 2006. The entire disclosure of said provisional application is hereby incorporated herein by reference, for all purposes.

### GOVERNMENT RIGHTS IN INVENTION

Work related to the invention hereof was conducted in the performance of Grant No. NIH/NIGMS GM066287 awarded by the U.S. National Institutes of Health. The government has certain rights in this invention.

### BACKGROUND OF THE INVENTION

Expansion of a repeating trinucleotide sequence in the genome above a certain length has been linked to the manifestation of several human disorders. These repeat disorders can be subdivided into expansions that occur in the noncoding sequence, such as introns or untranslated portions of mRNAs, or in the coding sequence (1, 2). Because amino acids are encoded by codons composed of three nucleotides, the resulting translation of a sequence with a trinucleotide repeat generates a protein with a repeating amino acid. So far most trinucleotide repeats that occur in the coding sequence are translated into a homomeric stretch of either glutamine or alanine amino acids. Expanded polyglutamine tracts have been found in nine different proteins that when mutated, cause several different neurodegenerative disorders (3, 4). Coincidentally, expanded polyalanine tracts have been found in nine different proteins, all of which are transcription factors, with one exception, a protein that binds the polyA nucleotide that is frequently present at the end of most mRNAs (reviewed in 5-7). Because all of the proteins containing polyalanine expansion are involved in global regulation of RNA functions of many important genes it is perhaps not surprising that diseases associated with expanded polyalanine proteins are associated with congenital deformities of different parts of the body.

The mechanisms by which expanded polyglutamine and polyalanine proteins cause disease is still not known, although studies conducted so far suggests that the expansions induce disease by a dominant gain and not loss-of-function (8). This conclusion is based on the fact that animals that are disrupted in the genes that are subject to expansions do not recapitulate many of the disease symptoms or pathology associated with the expansions, whereas transgenic expression of genes or cDNAs encoding all, or part, of the protein containing the expansions frequently recapitulates many of the disease symptoms (8-10).

Because polyalanine and polyglutamine disorders involve different amino acids it is instructive to know whether the diseases caused by the two different amino acids have any similarities. A comparison of the proteins in the pathology of expanded polyglutamine and polyalanine proteins has revealed two particular notable similarities, an amino acid length-dependent induction of protein aggregation and cell death (11-13). There are 9 different human disorders caused by expansion of polyalanine tracts in proteins.

It would be a significant advance in the art to provide the capability for suppressing the protein aggregation and cell death effects that have been associated with the pathology of polyalanine disorders.

### SUMMARY OF THE INVENTION

The present invention relates to the use of ubiquilin to reduce polyalanine protein aggregates and cell death. The invention is based on the discovery that overexpression of ubiquilin can clear polyalanine aggregates and reduce cell death and that overexpression of ubiquilin is useful in clearing misfolded protein aggregates from accumulating in cells. The invention therefore contemplates methods to modulate ubiquilin expression having utility to treat diseases not only associated with expanded polyalanine and polyglutarnine proteins, but also diseases associated with misfolding and aggregation of other unrelated proteins.

In one embodiment, the invention relates to a method of controlling ubiquilin expression levels as a means to regulate toxicity and cell death induced by expanded polyalanine proteins. In another embodiment, ubiquilin expression levels are regulated in order to prevent toxicity induced by expanded polyalanine proteins. In yet another embodiment, ubiquilin is utilized to rid cells of polyalanine aggregates.

The invention encompasses the use of ubiquilin to prevent or cure diseases caused by expansion of polyalanine proteins in various implementations, including using methods to increase ubiquilin levels in order to reduce accumulation of polyalanine aggregates and toxicity. Methods that can be utilized to increase ubiquilin levels include, without limitation: expression and use of cDNAs and genes encoding human ubiquilin proteins; expression and use of ubiquilin homologs from other species including C. elegans; introduction of ubiquilin protein into cells; and use of drugs and agents that induce ubiquilin levels that are effective for treatment or prophylaxis of disease states and conditions that are caused by expansion of polyalanine proteins.

In one aspect, the invention relates to a method for decreasing cell death in a cell exhibiting aggregation of polyalanine-containing proteins, the method comprising:

introducing an expression vector to a host cell comprising a nucleotide sequence encoding ubiquilin; and
maintaining the transformed host cell under biological conditions sufficient for expression and accumulation of the ubiquilin in the host cell, wherein overexpression of ubiquilin reduces sensitivity of cell stress induced by expanded polyalanine proteins.

In one embodiment of such method, the expression vector comprises a nucleotide sequence that encodes polypeptides comprising the amino acid residue of ubiquilin, or variants having at least 90% homology and having the same functional activity of ubiquilin, or fragments thereof.

In another embodiment of such method, the nucleotide sequence is selected from among SEQ ID Nos: 1, 3, 5, 9, 11, and 13.

The invention relates in another aspect to a method for determining the effectiveness of ubiquilin in reducing polyalanine expansion in a host cell, the method comprising:

introducing an expression vector to a host cell comprising a nucleotide sequence encoding ubiquilin; maintaining the transformed host cell under biological conditions sufficient for expression and accumulation of the ubiliquilin in the host cell; and
measuring the level of cell death in the host cells relative to a host cell not expressing increased levels of ubiliquin.

A further aspect of the invention relates to a method of treatment of disease associated with expanded polyalanine and polyglutamine proteins, or disease associated with misfolding and aggregation of unrelated proteins, or a neurological disorder, comprising administration, to a subject afflicted therewith, of an expression vector encoding for ubiquilin protein or variant thereof having deletions or substitution but maintaining the functionality of ubiquilin.

In one embodiment of such method, the neurological disorder comprises Huntington's disease.

The invention also contemplates a method of reducing polyalanine protein aggregates and cell death, comprising overexpressing ubiquilin in a cellular locus susceptible to such aggregates and cell death.

Another aspect of the invention relates to a method of clearing misfolded protein aggregates from accumulating in cells in a cellular locus, comprising overexpressing ubiquilin in said cellular locus.

A further aspect of the invention relates to a method of treating disease associated with expanded polyalanine proteins in a cellular locus, comprising overexpressing ubiquilin in said cellular locus.

Yet another aspect of the invention relates to a method of preventing toxicity induced by expanded polyalanine proteins in a cellular locus, comprising overexpressing ubiquilin in said cellular locus.

Other aspects, features and embodiments of the invention will be more fully apparent from the ensuing disclosure and appended claims.

### BRIEF DESCRIPTION OF THE DRAWINGS

FIG. 1. Ubiquilin associates with expanded GFP-polyalanine proteins.

A. Representative images of HeLa cells transfected with either a GFP-A7 or GFP-A37 expression plasmid alone or together with a ubiquilin-1 cDNA expression plasmid. 24 hours after the transfection, cells were fixed and immunostained for ubiquilin. The images shown in each row were captured by confocal microscopy and show the ubiquilin (abbreviated as Ubqln in all subsequent figures, red) and GFP (green) fluorescent images taken through a group of transfected cells, and the resulting image produced from merging the red and green images. Please note that the nuclei of GFP-positive cells contained brighter anti-ubiquilin staining (indicated by arrows) than nuclei of adjacent presumably untransfected cells. Bar, 20 im for all the panels.

B. Higher magnification images of the cells cotransfected with GFP-A37 and ubiquilin-1 cDNA (low panels), as described above. Bar, 5 im.

C. More ubiquilin coimmunoprecipitates with GFP-A37 than GFPA7 or GFP proteins. HeLa cells were transiently transfected with GFP, GFP-A7, or GFP-A37 constructs and lysates were prepared from the cells and the GFP expressed proteins were immunoprecipitated from them using a polyclonal anti-GFP antibody. The immunoprecipitated complexes were separated by SDS-PAGE, the proteins transferred to nitrocellulose membranes, and then immunoblotted (TB) with monoclonal antibodies against ubiquilin (upper panel), GFP (middle panel), or ubiquitin (lower panel).

D. Ubiquilin is present with GFP-A37 aggregates trapped on filters. Cell lysates were prepared from GFP-A7- and GFP-A37-transfected HeLa cells and filtered through a cellulose acetate membrane to trap protein aggregates. The filter membrane was first immunoblotted with a anti-ubiquilin monoclonal antibody and after stripping was then re-blotted with a anti-GFP polyclonal antibody.

FIG. 2. Overexpression of ubiquilin-1 cDNA reduces GFP-polyalanine protein-induced cell death.

A. HeLa cells were transfected with I ig of GFP, GFP-A7, or GFP-A37 expression plasmids alone or together with a ubiquilin-1 expression plasmid. The next day, the cells were treated with 100 tM of H,O, for 6 hours after which cell death was quantified by doubly staining the cells with Hoechst or propidium iodide (PT). Fragmented nuclei and PT-positive stained cells were counted as dead cells. The results show that overexpression of ubiquilin-1 reduces GFP-A37 induced cell death. * p<0.05.

B. Overexpression of ubiquilin-1 cDNA reduces GFP-A37-induced cell death in a dose-dependent manner. HeLa cells were transfected with 1 ig of GFP-A37 construct along with the indicated amounts of ubiquilin-1 cDNA and the amount of cell death was quantified as described above.

FIG. 3. Overexpression of ubiquilin-1 cDNA reduces the amount of GFP-polyalanine containing aggregates in cells.

A. HeLa cells were cotransfected with GFP-A7 or GFP-A37 expression plasmids and either an empty vector plasmid or a ubiquilin cDNA-expression plasmid. 24 hours after transfection, the cells were lysed and the amount of GFP-containing protein aggregates in similar amounts of protein lysate was determined by the filter trap assay (bottom panel). Meanwhile, equal portions of the lysates were immunoblotted for ubiquilin, GFP, and actin proteins.

B. Representative images (low and high magnification) of GFP-A37 aggregates seen in HeLa cells that were transfected with the GFP-A37 construct alone. Bar, 5 im.

C. Visual counting of HeLa cells transfected with GFP-A37 or GFP-A37 and ubiquilin-1 cDNA showing the proportion of GFP-fluorescent cells in which obvious aggregates were seen. By this analysis, ubiquilin-1 overexpression significantly reduced the amount of GFP-aggregates found in cells. * p<0.001.

D. Biochemical analysis demonstrating that overexpression of ubiquilin-1 cDNA reduces the amount of GFP-A37-containing protein aggregates in cells in a dose-dependent manner. HeLa cells were co-transfected with GFP-A37 and an increasing amount of ubiquilin-1 cDNA expression plasmid or an equivalent amount of empty vector plasmid as indicated. 24 hours after transfection, the cells were lysed and analyzed for the presence of GFP-containing aggregates or ubiquilin, GFP, or actin proteins as described in A above.

FIG. 4. Overexpression of ubiquilin-1 cDNA protects HeLa cell lines stably expressing expanded polyalanine proteins against increased vulnerability to H,02-induced cell death.

A. A GFP immunoblot of equivalent amount of protein lysate from three stable cell lines showing equivalent expression of either GFP alone, GFP-A7 or GFP-A37 proteins.

B. Representative fluorescent images of the GPP-, GFP-A7-, and GFP-A37-expressing stable cell lines used in A as well as in studies described below. Bar, 5 am.

C. The GFP-A7 and GFP-A37 cell lines were challenged with 100 1 aM of H,O, for 5 hours and then stained with Hoechst 33343 and P1 to determine the extent of cell death in the cultures. An additional set of the cultures was transfected with the ubiquilin-1 cDNA prior to the H,02 treatment. The graphs show that the increased vulnerability of the GFP-A37 expressing cells to H₂0₂-induced cell death is partially attenuated by overexpression of ubiquilin-1. Cell death was quantified as described in the methods. * p<0.05.

D. Biochemical analysis showing overexpression of ubiquilin-1 reduces the amount of GFP-containing aggregates in the GFP-A37 cell line. The GFP-A7 and GFP-A37 cell lines were transferred with either a ubiquilin-1 expression plasmid or the empty vector. After 24 hours, the cells were lysed and the amount of GFP-immunoreactive aggregates present in equal protein portions of the lysates was determined by the filter trap assay (bottom panel). Meanwhile, equal amounts of the protein lysates were also immunoblotted for ubiquilin, GFP, and actin (upper three panels).

E. Biochemical analysis demonstrating overexpression of ubiquilin-1 cDNA reduces the amount of GFP-A37-containing protein aggregates in cells in a dose-dependent manner. The GFP-A37 cell line was transfected with varying amounts of ubiquilin-1 cDNA expression plasmid (0 to 0.9 μg DNA), or an equivalent amount of empty vector plasmid as indicated. 24 hours after transfection, the cells were lysed and analyzed for the presence of GFP-containing aggregates or ubiquilin, GFP, or actin proteins, similar to D.

FIG. 5. Reduction of ubiquilin expression by RNAi in the GFP-A37 cell line leads to a decrease in cellular proliferation and increases in DNA fragmentation and cell death.

A. Ubiquilin and actin immunoblots of equal amounts of protein lysates from cultures of the GFP-A37 cell line that were transfected with a combination of siRNAs specific for ubiquilin-1 and ubiquilin-2, or with control siRNAs that do not target any known gene, or mock-transfected. Bar, 100 μm.

B. Representative phase contrast images showing the equivalent cell density of the three groups of cells at the beginning of a similar experiment described in A. Bar, 100 tm.

C. Representative GFP and Hoechst fluorescence images of the experiment described in B at four days after transfection. Note the decrease in proliferation and increase in nuclear condensation in the cells transfected with ubiquilin siRNAs.

D. Quantification of nuclear fragmentation in the experiment described in B and C. * p<0.0001.

E. Quantification of cell death in the experiment described in B and C. * p<0.01.

FIG. 6. Reduction of ubiquilin expression by RNAi in the GFP-A37 cell line results in increased accumulation of GFP-containing aggregates in cells.

A. Similar experiment as described in FIG. 5 showing high magnification of representative GFP fluorescence images of cells four days after transfection. Note the brighter fluorescence of GFP aggregates in the cells transfected with ubiquilin siRNAs (indicated by arrows). Bar, 5 im.

B. A GFP immunoblot of a filter trap assay to measure protein aggregates present in equal amounts of protein lysate (10, 20 or 40 μg) prepared from mock, ubiquilin siRNA, and control siRNA transfections of the GFP-A37 cell line. Note the increase in the amount of aggregates in the lysates from the cells that were transfected with ubiquilin siRNAs.

### DETAILED DESCRIPTION OF THE INVENTION, AND PREFERRED EMBODIMENTS THEREOF

The present invention relates to the use of ubiquilin to reduce polyalanine protein aggregates and cell death, and reflects our discovery that ubiquilin can reduce protein aggregates and cytotoxicity of proteins containing polyalanine expansions.

Several human disorders are associated with an expansion of a continuous stretch of alanine amino acids in proteins. These so-called polyalanine expansion diseases are characterized by a length-dependent reiteration of amino acid induction of protein aggregation and cytotoxicity. Unlike polyglutamine disorders, in which the number of glutamines can fluctuate rapidly, and sometimes exceed over 100, polyalanine-related disease are associated with smaller expansions, and frequently expansions of only two residues above a threshold of 20 is sufficient to cause disease. Overexpression of ubiquilin can be employed to effectively reduce protein aggregates and toxicity of expanded polyalanine proteins.

In one aspect, the present invention relates to a method for decreasing cell death in a cell exhibiting aggregation of polyalanine-containing proteins. The method includes introducing an expression vector to a host cell comprising a nucleotide sequence encoding ubiquilin, and maintaining the transformed host cell under biological conditions sufficient for expression and accumulation of the ubiquilin in the host cell, wherein overexpression of ubiquilin reduces sensitivity of cell stress induced by expanded polyalanine proteins.

In such method, the expression vector can comprise a nucleotide sequence that encodes polypeptides comprising the amino acid residue of ubiquilin, or variants having at least 90% homology and having the same functional activity of ubiquilin, or fragments thereof.

In another embodiment of such method, the nucleotide sequence is selected from among SEQ ID Nos: 1, 3, 5, 9, 11, and 13 of the accompanying sequence listing.

The invention in a further specific aspect relates to a method for determining the effectiveness of ubiquilin in reducing polyalanine expansion in a host cell, by introducing an expression vector to a host cell comprising a nucleotide sequence encoding ubiquilin, maintaining the transformed host cell under biological conditions sufficient for expression and accumulation of the ubiliquilin in the host cell, and measuring the level of cell death in the host cells relative to a host cell not expressing increased levels of ubiliquin.

A further aspect of the invention relates to a method of treatment of disease associated with expanded polyalanine and polyglutamine proteins, or disease associated with misfolding and aggregation of unrelated proteins, or a neurological disorder, e.g., Huntington's disease, comprising administration, to a subject afflicted therewith, of an expression vector encoding for ubiquilin protein or variant thereof having deletions or substitution but maintaining the functionality of ubiquilin.

Overexpression of ubiquilin-1 in HeLa cells is demonstrated herein to reduce protein aggregates and the cytotoxicity associated with expression of a transfected nuclear-targeted GFPfusion protein containing 37-alanine repeats (GFP-A37), in a dose dependent manner. Ubiquilin coimmunoprecipitated more with GFP proteins containing a 37-polyalanine tract compared to either 7 (GFP-A7), or no alanine tract (GFP) Moreover, overexpression of ubiquilin suppressed the increased vulnerability of HeLa cell lines stably expressing the GFP-A37 fusion protein to oxidative stress-induced cell death compared to cell lines expressing G-FP or GFP-A7 proteins. By contrast, siRflA knockdown of ubiquilin expression in the GTP-A37 cell line was associated with decreased cellular proliferation, and increases in GEP protein aggregates, nuclear fragmentation, and cell death. These results indicate that boosting ubiquilin levels in cells is a useful and attractive strategy to prevent toxicity of proteins containing reiterative expansions of amino acids involved in many human diseases.

### Results

Ubiquilin Binds and Colocalizes with GFP-Proteins Containing Expanded Polyalanine Tracts

To test whether ubiquilin can reduce polyalanine-induced protein aggregates and toxicity we utilized two previously characterized expression constructs that have been used to model polyalanine protein-aggregation and toxicity in cells and organisms (11, 13, 15, 16). The constructs encode GFP fused with either 7 or 37 consecutive alanine amino acids, plus a SV4O nuclear localization signal, henceforth referred to as GFP-A7 and GFP-A37, respectively. The NLS was incorporated in the constructs because all of the human proteins containing polyalanine expansions are thought to localize and function in the nucleus. Previous studies had shown that expression of the GFP-A37-fusion protein in cells and organisms leads to a dose-dependent increase in GFP protein aggregation as well as an increase in cell death compared to expression of the GFP-A7 fusion protein (15).

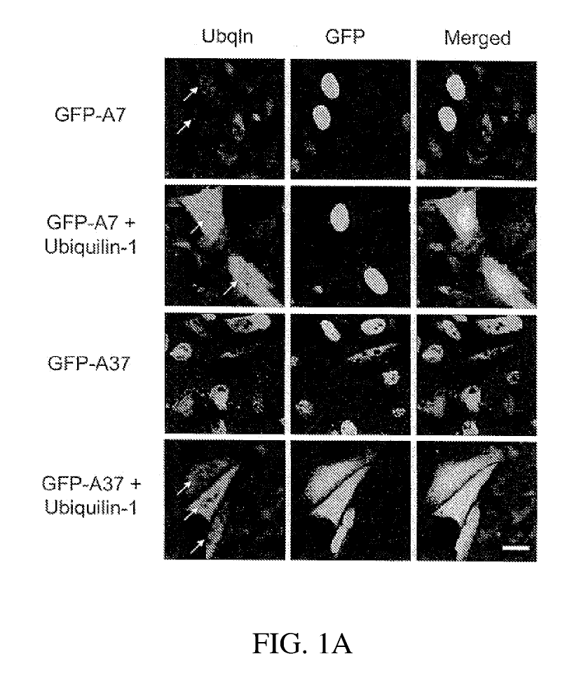

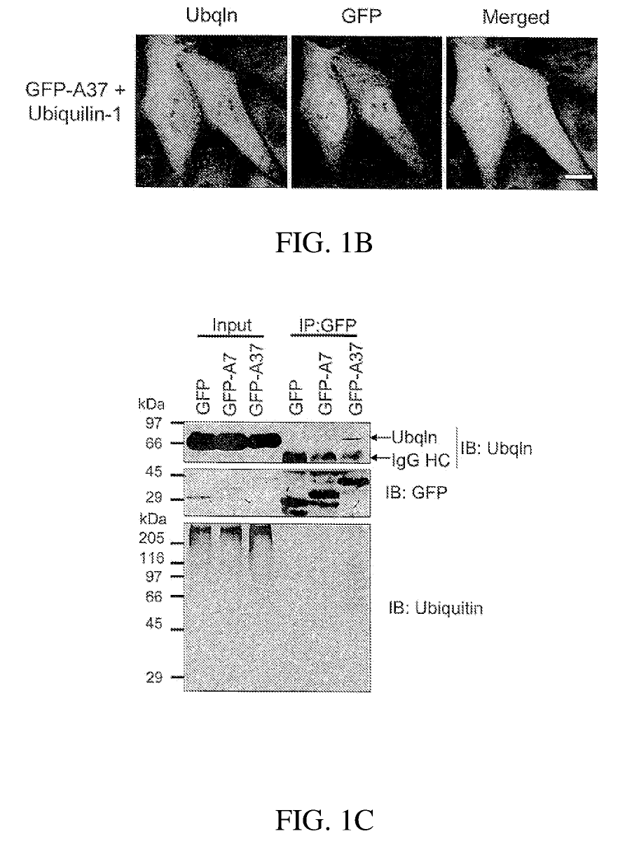

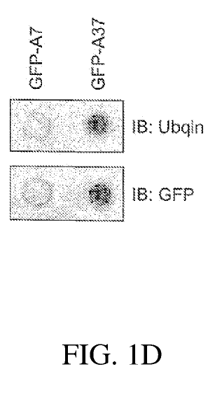

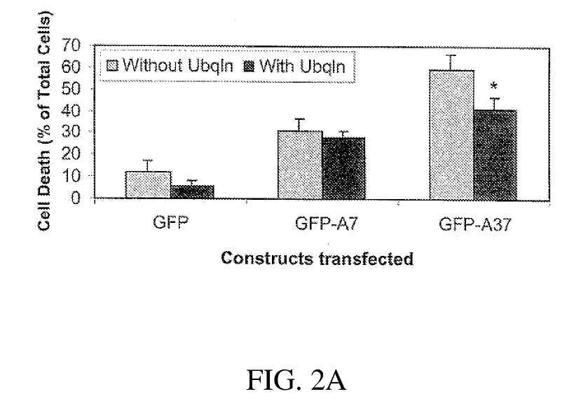

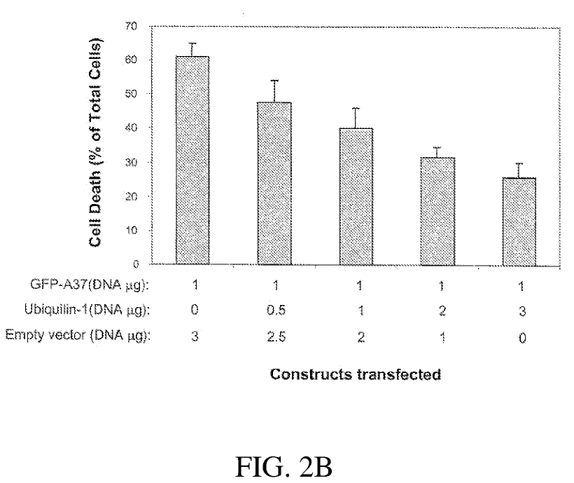

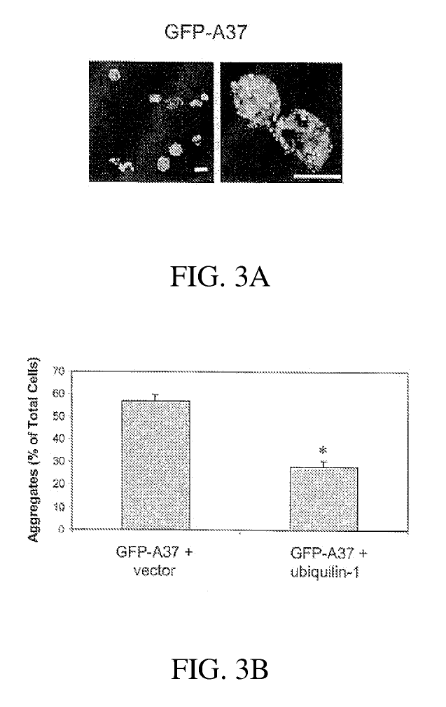

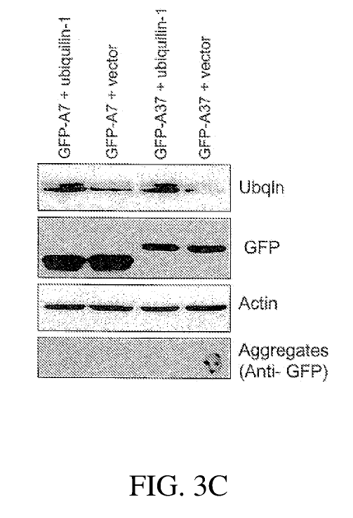

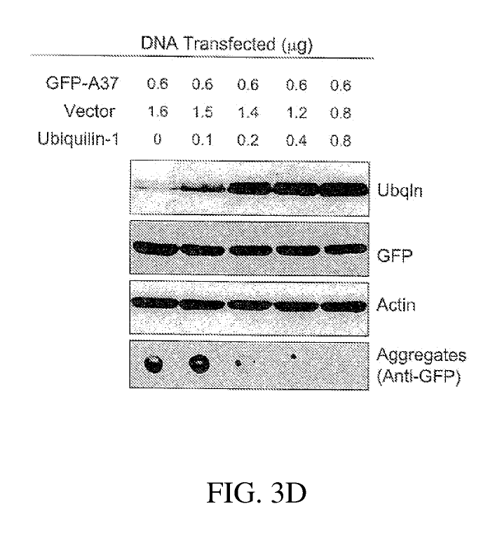

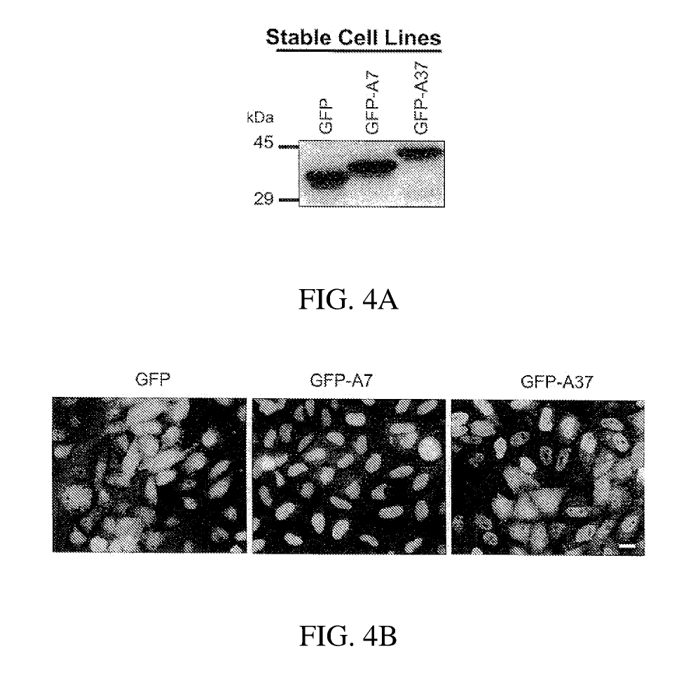

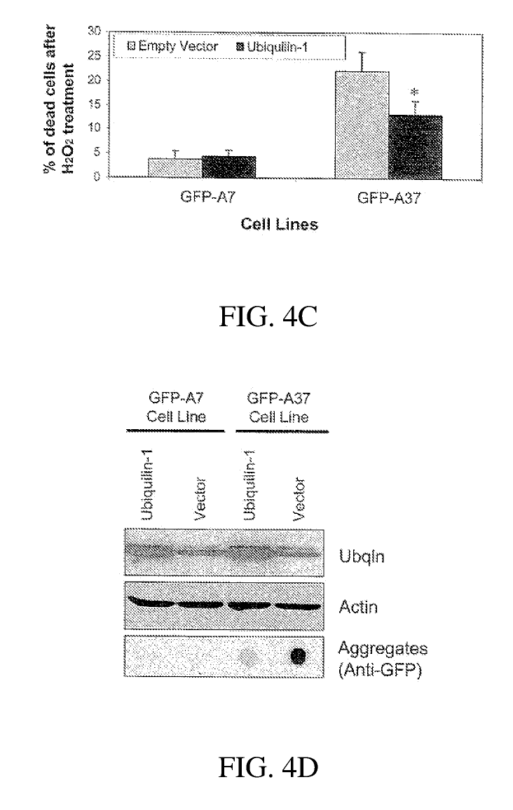

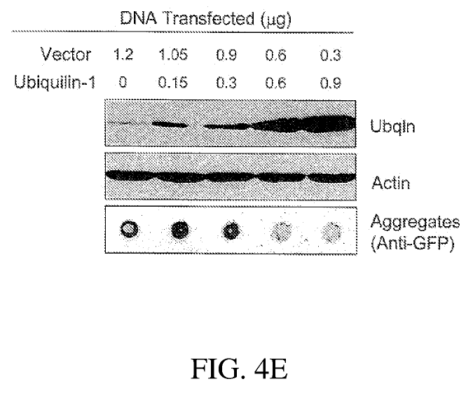

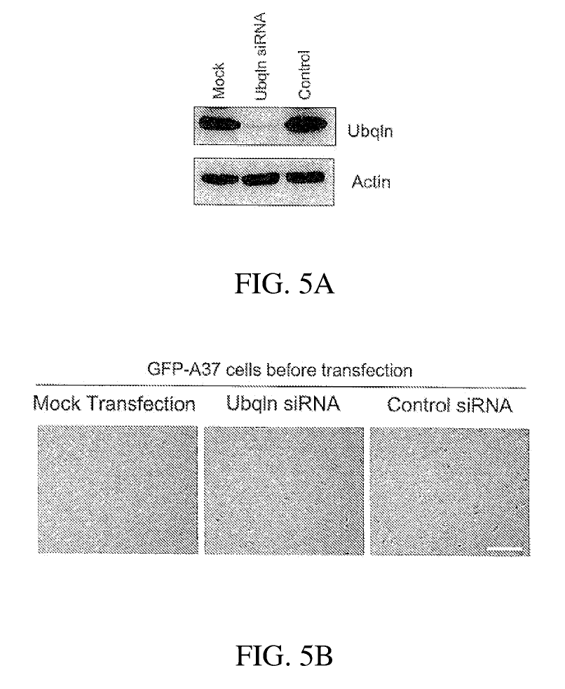

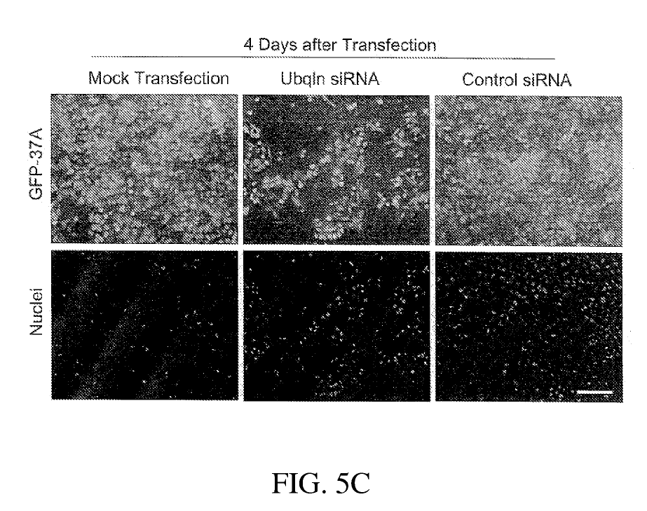

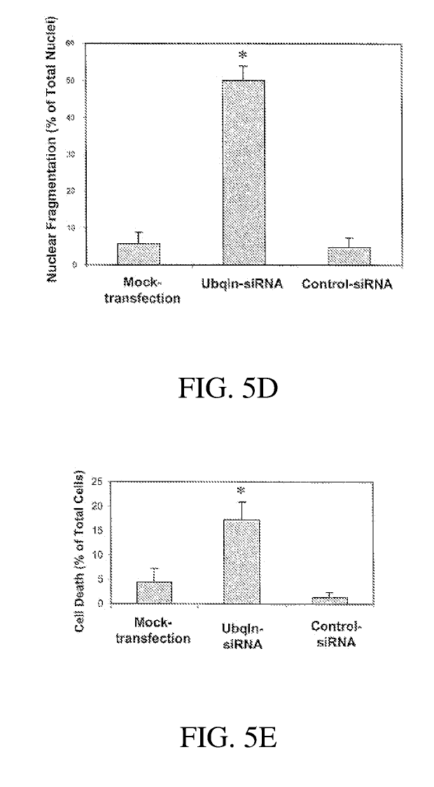

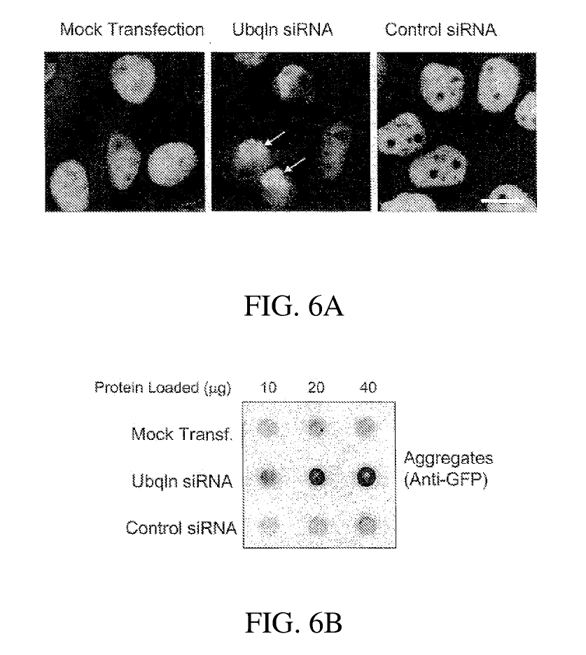

To determine how alterations in ubiquilin protein levels affects aggregation and toxicity of polyalanine-containing proteins, GFPA7 and GFPA37 expression constructs were expressed in HeLa cells, together with or without a human ubiquilin-1 cDNA expression plasmid. HeLa cells were utilized because they are of human origin and because protocols were established for both overexpression as well as knockdown of human ubiquilin proteins (14). As shown in FIG. 1A, confocal microscopy of HeLa cells that were transfected with either the GFP-A7 expression plasmid alone (first panel) or cotransfected with ubiquilin-1 expression plasmid (second panel) revealed strong and almost uniform anti-GFP staining predominantly in the nucleus. The morphology the cells overexpressing GFP-A7, either alone or together with ubiquilin-1, were similar to that of untransfected cells. By contrast HeLa cells transfected with GFP-A37 alone displayed visible GFP-fluorescent aggregates in both the cytoplasm and nucleus, but many of the nuclei and cells had a shrunken and rounded-up morphology, respectively (FIG. 1A third panel). Interestingly, these abnormal morphologies were not apparent in cells cotransfected with GFP-A37 and ubiquilin-1 expression plasmids (fourth panel), despite clear indication that the GFP-A37 protein was overexpressed, supporting the conclusion that overexpression of ubiquilin can prevent manifestation of these abnormal morphologies.

Examination of GFP and anti-ubiquilin staining by double immunofluorescence microscopy in the cells cotransfected with GFP-A7 and ubiquilin-1 or GFP-A37 and ubiquilin-1 expression constructs revealed a clear increase in ubiquilin immunoreactivity compared to the presumably non-transfected cells. The increase in ubiquilin staining in these cotransfected cells was present throughout the cytoplasm and nucleus with the exception of oval structures in the nucleus, which were presumed to be nucleoli (FIG. 1B). Interestingly, in the GFP-A37 overexpressing cells, the patterns of ubiquilin and GFP staining in the cytoplasm colocalized well with one another suggesting possible interaction of the proteins. Ubiquilin staining also colocalized with the GFP-A7 and GFP-A37 fusion proteins in the nucleus (FIG. 1A), but because the proteins displayed somewhat uniform staining in this organelle it was difficult to determine if it arose by fortuitous overlap of two proteins or from specific interaction between the proteins. A hint that ubiquilin might indeed colocalize with the GFP expressed polyalanine fusion proteins derived from the fact that cells transfected with either GFP-A7 or GFP-A37 expression constructs alone contained increased accumulation of endogenous ubiquilin in the nucleus compared to that in the non-GFP expressing cells (FIG. 1A indicated by arrows).

To determine if ubiquilin interacts with GFP-A7 or GFP-A37 fusion proteins, GFP expressed proteins from cells that were either singly transfected with GFP, or GFP-A7, or GFP-A37, expression constructs were immunoprecipiated and immunoblotted for ubiquilin. As shown in FIG. 1C more ubiquilin coimmunoprecipitated with GFP-A37 than with either GFPA7 or GFP proteins. Because ubiquilin is known to bind polyubiquitinated proteins, it appeared that more ubiquilin coimmunoprecipitated with GFP-A37 than GFP-A7 protein because the former is more prone to aggregate and to be ubiquitinated. Consistent therewith, more anti-ubiquitin immunoreactivity was detected in the GFP-A37 immunoprecipitated proteins than with the GFP-A7 or GFP proteins (FIG. 1C). Furthermore, a filter trap assay used to measure protein aggregates in cell lysates revealed that cells transfected with GFP-A37 to contained more GFP- and ubiquilin-immunoreactive aggregates than cells transfected with the GFP-A7 construct (FIG. 1D). These results suggested that ubiquilin can interact more strongly with GFP-expressed proteins with longer polyalanine tracts.

### Overexpression of Ubiquilin in Hela Cells Reduces the Amount of GFP-Polyalanine Aggregates and Cytotoxicity

To compare the cytotoxic properties of GFP-A7 and GFP-A37 fusion proteins, nuclear fragmentation and cell death of HeLa cells transfected with the two expression constructs was measured. As shown in FIG. 2A, expression of the GFP-A37 construct correlated with a higher percentage of GFP-expressing cells that exhibited nuclear fragmentation and death properties compared to expression of either GFP-A7, or GFP alone. The differential cytotoxic property of the two constructs in HeLa cells is in accord with the greater cytotoxic properties of the GFP-A37 found in other cell types (15). Next, overexpression of ubiquilin-1 was investigated to determine if it might suppress the toxicity induced by the polyalanine proteins. As shown in FIG. 2B, coexpression of ubiquilin-1 cDNA with GFP-A37 reduced the GFP-A37-induced cell death in a dose-dependent manner. By contrast, there was negligible, if any, reduction, in the extent of nuclear fragmentation and cell death in cells cotransfected with ubiquilin-1 cDNA and GFP-A7 (FIG. 2A). These results support the conclusion that ubiquilin overexpression can selectively suppress the toxicity of polyalanine containing proteins with a repeat length known to cause disease.

It was next determined whether the protective effect of ubiquilin towards GFP-A37-induced cytotoxicity correlated with a change in polyalanine protein aggregation. To examine this possibility, cell lysates prepared from cells transfected with either GFP-A7 or -A37 expression constructs alone, or together with ubiquilin-1 cDNA, were immunoblotted for the presence of GFP aggregates trapped on filters (FIG. 3A). By this assay GFP-immunoreactive protein aggregates were only detected in the cells that were singly transfected with GFP-A37 but not GFP-A7. Importantly, the amount of these GFP-containing aggregates was reduced in cells that were cotransfected with GFP-A37 and ubiquilin-1 constructs (FIG. 3A). The reduction of protein aggregates scored by this biochemical approach correlated well with a reduction in visible GFP-fluorescent aggregates seen in cells cotransfected with ubiquilin-1 and GFP-A37 compared to cells transfected with GFP-A37 alone (FIGS. 3B and C). Furthermore, the reduction in GFP-A37 protein aggregation modulated by ubiquilin-1 appeared to be dependent on the amount of ubiquilin-1 expressed, because transfection of an increasing amount of ubiquilin-1 cDNA expression plasmid resulted in a dose-dependent reduction of GFP-A37 aggregates, as scored by the filter trap assay (FIG. 3D). The reduction in GFP-containing aggregates by ubiquilin was not simply due to decreased GFP-fusion protein expression, because immunoblots of equal amounts of protein from these experiments revealed that the GFP-fusion proteins were expressed to similar levels in the ubiquilin-transfected and non-transfected cells (see GFP panels in FIGS. 3C and 3D).

Together these results show that ubiquilin overexpression reduces the amount of GFP-A37 aggregates that accumulates in cells, and prevents the cytotoxicity observed upon expression of the expanded GFP-A37 protein in cells.

### Overexpression of Ubiquilin-1 Suppresses H₂O₂-Induced Cell Death of Stable Cell Lines Expressing Expanded Polyalanine Proteins

To obtain further evidence in support of the finding that the amount of ubiquilin expressed in cells modulates toxicity of proteins with expanded polyalanine tracts, HeLa cell lines that stably expressed either GFP alone, or GFP-A7-, or GFP-A37-fusion proteins, were isolated. Unlike transiently transfected cells where expression of the GFP-fusion proteins varied considerably, the stable cell lines expressed a constant amount of the proteins, providing a more reliable system for evaluating the toxicity of the polyalanine proteins. Lines that stably expressed comparable levels of each GFP protein, determined by immunoblotting, were selected for further studies (FIG. 4A). The experiments described below were repeated with other cell lines expressing the proteins and similar results to those described below were obtained. For simplicity purposes, data from only one set of these lines is presented. Similar to the pattern found in transiently transfected cells, the GFP-A7 and GFP-A37 expressing stable cell lines displayed GFP—fluorescence mainly in the nucleus, consistent with appropriate targeting of the proteins by the NLS that was incorporated into each polypeptide (FIG. 4B). Interestingly, the cell line expressing GFP-A37 contained higher levels of GFP fluorescence in the cytoplasm compared to the GFP-A7-expressing line, a phenotype that was also seen in transiently transfected cells (FIG. 4B). The reason for the greater sequestration of GFP-A37 protein in the cytoplasm compared to the GFP-A7 protein is not known but may be related to differences in aggregation and/or binding properties of the proteins.

Because it was found that cell lines that express proteins with expanded polyglutamine tracks are acutely more sensitive to agents that induce oxidative stress (14) than those that do not express the expanded proteins, GFP-A7- and GFP-A37 lines were studied to determine whether they would also be differentially vulnerable to such agents. To test this possibility the GFP expressing cell lines were treated with 100 μM H₂O₂ for 5 hours and it was found that the GFP-A37 line, but not the GPP-A7 or GFP cell lines, was acutely sensitive to exposure with this dose of H₂O₂ (FIG. 4C). Approximately 22% of the cells from the GPP-A37 line when exposed to H₂O₂ died (FIG. 4C), while the GFP-A7 and GFP expressing cells were robust when subjected to this same treatment (FIG. 4C and results not shown). To determine if increased ubiquilin expression can protect GFP-A37 cells against the H₂O₂ insult, cell death was measured in GFP-A7 and GFP-A37 cell lines that were first transfected with either a ubiquilin-1 cDNA expression plasmid or the empty plasmid vector and then exposed to H₂O₂. The percentage of dead cells in the GPP-A7 line was low and remained unaltered in the cells transfected with either the control vector or with the ubiquilin-1 cDNA (FIG. 4C). By contrast, there were approximately 40% fewer dead cells in the GPP-A37 cell line that were transfected with ubiquilin-1 cDNA compared to the vector control (FIG. 4C).

Lysates were also prepared from the transfected cells to examine if GPP-protein aggregation was altered in them using the filter trap assay. As shown in FIG. 4E, transfection of ubiquilin-1 cDNA, but not the empty vector, significantly reduced the amount of GFP aggregates in the GPP-A37 cell line. A similar reduction was observed in the GPP-A7 transfected cells (FIG. 4D), but this line contained significantly fewer aggregates to begin with, as expected. Further studies revealed that ubiquilin overexpression reduced GPP-polyalanine protein aggregation in the GFP-A37 cell line in a dose-dependent manner (FIG. 4E).

Together these results support the conclusion that overexpression of ubiquilin-1 can protect cell lines that express expanded polyalanine proteins from an increase in susceptibility to oxidative stress, which correlates with a reduction in accumulation of GFP-polyalanine-containing protein aggregates.

Reduction of Ubiquilin Protein Expression in GFP-A37 Cells Leads to an Arrest in Cellular Proliferation and Correlates with Increases in GFP Protein Aggregates, Nuclear Fragmentation and Induction of Cell Death

To confirm the role of ubiquilin in protecting cells against polyalanine toxicity, RNA interference (RNAi) was used to reduce ubiquilin protein levels in the GFP-A37 HeLa cell line to examine if reduction of its expression would increase polyalanine-induced protein aggregates and cell death. Because HeLa cells express two predominant ubiquilin isoforms, ubiquilin-1 and ubiquilin-2 (17), we transfected the GFP-A37 cells with a combination of siRNAs to specifically knockdown expression of both proteins. An immunoblot confirmed that both ubiquilin 1 and 2 proteins were indeed reduced by approximately 80 to 90%, respectively, compared to untransfected or mock-transfected cells (FIG. 5A). Knockdown of the ubiquilin expression in the GFP-A37 cells resulted in a dramatic arrest in cellular proliferation, which correlated with a high rate of nuclear fragmentation and cell death (FIG. 5B-E). Almost none of these phenotypes were observed in GFP-A37 cells that were either mock-transfected or transfected with control siRNAs that were designed not to induce genetic interference of any known gene.

Finally, a study was undertaken to determine if RNAi of ubiquilin expression altered GFP protein aggregation in the GFP-A37 cell line. Changes in GFP aggregation were analyzed by fluorescence microscopy and by the filter trap assay. It was noticed that the distribution of GFP fluorescence in the nucleus of GFP-A37 cells transfected with ubiquilin siRNAs had a more condensed distribution and formed brighter foci in the nucleus as compared to the uniform distribution of the protein in mock and control siRNA transfected cells (FIG. 6A). Furthermore, the filter trap assay revealed significantly more GFP-containing aggregates in lysates of the cells transfected with ubiquilin siRNAs compared to those in the two control transfections (FIG. 6B).

Together these results indicate that a reduction in ubiquilin protein expression in cells expressing expanded polyalanine proteins increases the amount of GFP protein aggregates in cells, which correlates with an arrest in cellular proliferation and increases in nuclear fragmentation and cell death.

### Discussion

The foregoing results demonstrate an inverse relationship between the amount of ubiquilin protein expressed in cells and the accumulation of protein aggregates and cytotoxicity of proteins containing expanded polyalanine tracts. Support for this conclusion is based on the evidence that increased expression of ubiquilin-1 protein reduces the amount of GFP-A37 protein aggregates as well as the cytotoxicity associated with expression of the GFP A37 fusion protein in HeLa cells. It has also been demonstrated that the converse is true: a reduction of ubiquilin levels in cells by RNAi increases the amount of GFP-A37 protein aggregates, which correlates with an increase in cell death.

It is remarkable that ubiquilin is able to suppress the cytotoxicity of proteins containing either expanded polyalanine or polyglutamine tracts (as we have shown previously, 14), considering that the two amino acids involved in these expansions (glutamine and alanine) are so different. A feature that was found to be common to the cytoprotection of ubiquilin against proteins with expanded polyalanine and polyglutamine tracts was the inverse relationship between the amount of ubiquilin expressed in cells and the amount of aggregates formed by the expanded proteins. In both cases it was found that increased ubiquilin expression reduced the amount and number of aggregates containing the expanded proteins in cells and this correlated with an alleviation of the cytotoxicity associated with the expanded proteins. In both cases too, it was found that a reduction of ubiquilin levels increased the amount and number of the aggregates containing the expanded proteins, which correlated with greater induction of cytotoxicity by the proteins.

The results presented herein are consistent with the notion that a build-up of aggregates composed of polyalanine and polyglutamine proteins is toxic to cells. In accord with the notion that aggregates are toxic, it was observed that expression of GFP-A37, containing a stretch of 37 alanines, was more prone to form aggregates than GFP-A7, containing a stretch of 7 alanines, and this correlated with the more deleterious property of the GFP-A37 protein in transiently transfected and stable HeLa cell lines. Furthermore, overexpression of ubiquilin reduced the amount of polyalanine and polyglutamine aggregates that build-up in cells, and this directly correlated with a reduction in the toxicity associated with expression of the expanded polyalanine and polyglutamine proteins in cells.

The foregoing results do not show how ubiquilin protects cells against toxicity induced by expanded polyalanine and polyglutamine proteins. However, based on the properties of ubiquilin proteins discovered so far, it may function in any of the following way(s). One possibility is that ubiquilin may recruit misfolded proteins, such as those containing expanded polyalanine and polyglutamine tracts, to the proteasome for degradation. This property would be in accord with the known ability of ubiquilin to bind ubiquitinated proteins and proteasome subunits via its C and N-terminal domains, respectively (22-25). Thus, overexpression of ubiquilin may accelerate the delivery of misfolded proteins to the proteasome and thereby enhance their clearance. Consistent with this theory it was found that ubiquilin coimmunoprecipitated more with ubiquitinated, and the presumably the more prone to misfold, GFP-A37 fusion protein than with the GFP-A7 or GFP proteins, and that this correlated with a reduction in GFP-A37 aggregates in cells that overexpressed ubiquilin. Another possibility is that ubiquilin may enhance clearance of polyalanine and polyglutamic aggregates by autophagy. This possibility is consistent with the fact that ubiquilin has been found to interact with mTor, a key regulator of autophagy (26). Because inhibition of mTor kinase activity activates autophagy, it may be that overexpression of ubiquilin leads to increased binding to mTor, which might prevent the kinase from binding its normal targets, or inactivate the kinase, or stimulate mTor degradation. A preliminary report has suggested that ubiquilin overexpression does not alter mTor kinase activity (26). Finally, ubiquilin may reduce polyalanine and polyglutamine-induced toxicity due to its ability to function as a molecular chaperone, in a complex with other proteins. Consistent with this idea, ubiquilin reacts with Stch (27), a heat shock protein possessing an ATPase domain, which may be involved in refolding the potentially toxic misfolded proteins containing expanded polyalanine and polyglutamine tracts. Ubiquilin has been shown to protect neurons and cells from injury induced by oxidative stress and hypoxia (28) (14). The foregoing results show that ubiquilin protects cells from increased vulnerability to oxidative stress caused by expression of expanded polyalanine proteins.

### Materials and Methods

### Cell Culture, DNA Transfection, Establishment of Stable Cell Lines, and Fluorescent Microscopy

HeLa cells were cultured in Dulbecco's modified Eagle's medium (DMEM) supplemented with 10% fetal bovine serum. Cells were transfected with plasmid DNAs using the calcium phosphate coprecipitation method. Stable cell lines expressing different GFP proteins were isolated by cotransfecting HeLa cells with pNeo together with pEGFP, or with pEGFP-A7, or with pEGFP-A37 expression plasmids at a 1:10 ratio of the plasmids, respectively. After several days of selection with 0418 (700 Ig/ml) individual clones with GFP fluorescence were identified and expanded. Fluorescent images of fixed or live cells were captured using either a LSM5 10 laser scanning confocal microscope (Zeiss) equipped with an argon and two HeNe lasers or a Zeiss Axiovert 100 fluorescence microscope.

### Plasmid Constructs, SUS-PAGE, Filter Trap Assay, Immunoblotting, and Antibodies

GFP-A7 and GFP-A37 expression plasmids, which contain a nuclear localization signal (NLS), were provided by Dr. David C. Rubinsztein (University of Cambridge, UK). The construction of the ubiquilin-1 expression cDNA plasmid (30), protocols for SDSPAGE, immunoblotting and the filter trap assay (14), and GFP polyclonal and ubiquilin monoclonal antibodies (14, 17) are variously described in the literature.

### Quantification of Cell Death

Cell death was quantified in the cultures by counting the proportion of cells that exhibited an abnormal nuclear morphology under the microscope after staining of the cells with the DNA dye Hoechst 33342 (1 g/ml). Alternatively, cell death was quantified by counting the number of cells whose membrane permeability barrier to staining with 3 1 tM propidium iodide (P1) had been destroyed. Sensitivity of cells to H₂0₂ was performed as described previously (14).

### Knockdown of Ubiquilin Expression by RNA Interference

Expression of ubiquilin proteins were knocked down by transfecting cells with a 10 nM mixture of SMARTpool siRNAs directed specifically against human ubiquilin-1 and ubiquilin-2 sequences using a previously described protocol (14, 17). The stable GFP-A37 line was plated in 24-well plates (Costar) and 24 hours after the plating, the cultures were transfected with SMARTpools of siRNAs against either ubiquilin-1 and -2, or with control siRNAs that have no known target, or were mock transfected with the transfection reagent alone. The cultures were maintained for 4 days in the transfection medium and then cell death was quantified as described above or the cells were lysed and analyzed for either ubiquilin expression or for the presence of GFP protein aggregates by immunoblotting.

### Statistical Analysis

For statistical analysis, one-way analysis of variance (ANOVA) was applied. Significant variance between groups was determined using the t-test. Data are shown as mean±SDM and p<0.05 was considered statistically significant.

### BIBLIOGRAPHY

1. Pearson, C. E., Nichol Edamura, K. and Cleary, J. D. (2005) Repeat instability: mechanisms of dynamic mutations. Nat Rev Genet, 6, 729-42.
2. Gatchel, J R. and Zoghbi, H. Y. (2005) Diseases of unstable repeat expansion: mechanisms and common principles. Nat Rev Genet, 6, 743-55.
3. Bates, G. (2003) Huntington aggregation and toxicity in Huntington's disease. Lancet, 361, 1642-4.
4. Riley, B. E. and On, H. T. (2006) Polyglutamine neurodegenerative diseases and regulation of transcription: assembling the puzzle. Genes Dev, 20, 2183-92.
5. Brown, L. Y. and Brown, S. A. (2004) Alanine tracts: the expanding story of human illness and trinucleotide repeats. Trends Genet, 20, 5 1-8.
6. Amid, I., Trochet, D., Clement-Ziza, M., Munnich, A. and Lyonnet, S. (2004) Polyalanine expansions in human. Hum Mol Genet, 13 Spec No 2, R235-43.
7. Albrecht, A. and Mundlos, S. (2005) The other trinucleotide repeat: polyalanine expansion disorders. Curr Op/n Genet Dev, 15, 285-93.
8. Landles, C. and Bates, G. P. (2004) Huntington and the molecular pathogenesis of Huntingtons disease. Fourth in molecular medicine review series. EMBO Rep, 5, 958-63.
9. Bates, G. P. and Hockly, F. (2003) Experimental therapeutics in Huntington's disease: are models useful for therapeutic trials? Curr Op/n Neurol, 16, 465-70.
10. Michalik, A. and Van Broeckhoven, C. (2003) Pathogenesis of polyglutamine disorders: aggregation revisited. Hum Mol Gene, 12 Spec No 2, R173-86.
11. Rankin, J., Wyttenbach, A. and Rubinsztein, D. C. (2000) Intracellular green fluorescent protein-polyalanine aggregates are associated with cell death. Biochenz J, 348 Pt 1, 15-9.
12. Nasrallah, T M., Minarcik, J. C. and Golden, J. A. (2004) A polyalanine tract expansion in Arx forms intranuclear inclusions and results in increased cell death. J Cell Biol, 167, 411-6.
13. Berger, Z., Davies, I. E., Luo, S., Pasco, M. Y., Majoul, I., OKane, C. J. and Rubinsztein, D. C. (2006) Deleterious and protective properties of an aggregate-prone protein with a polyalanine expansion. Hum Mol Gene, 15, 453-65.
14. Wang, H. Lirn, P. I., Yin, C., Rieckher, M., Vogel, B. E. and Monteiro, M I. (2006) Suppression of polyglutamine-induced toxicity in cell and animal models of Huntington's disease by ubiquilin. Hum Mol Gene, 15, 1025-41.
15. Bao, Y. P., Cook. L. J., ODonovan, D., Uyarna, E. and Rubinsztein, D. C. (2002) Mammalian, yeast, bacterial, and chemical chaperones reduce aggregate formation and death in a cell model of oculopharyngeal muscular dystrophy. J Biol Diem, 277, 12263-9.
16. Berger, Z, Ravikumar, B., Menzies, F. M., Oroz, L. G., Underwood, B R., Pangalos, M N., Schmitt, I., Wuilner, U., Evert, B. O., OKane, C. J. et al. (2006) Rapamycin alleviates toxicity of different aggregate-prone proteins. Hum Mol Genet, 15, 433-42.
17. Massey, L. K., Mab, A, L. and Monteiro, M. J. (2005) Ubiquilin regulates presenilin endoproteolysis and modulates gamma-secretase components, Pen-2 and nicastrin. Biochemj, 391, 513-25.
18. La Spada, A. R. and Taylor, J. P. (2003) Polyglutamines placed into context. Neuron, 38, 68 1-4.
19, Ross, C. A. and Poirier, M A. (2004) Protein aggregation and neurodegenerative disease. Nat Med, 10 Suppl, S 10-7.
20. Arrasate, M., Mitra, S., Schweitzer, E. S., Segal, M. R. and Finkbeiner, S. (2004) Inclusion body formation reduces levels of mutant huntington and the risk of neuronal death. Nature, 431, 805-10.
21. Bowman, A. B., Yoo, S. Y., Dantuma, N. P. and Zoghbi, H. Y. (2005) Neuronal dysfunction in a polyglutarnine disease model occurs in the absence of ubiquitinproteasome system impairment and inversely correlates with the degree of nuclear inclusion formation. Hum Mol Genet, 14, 679-91.
22. Massey, L. K., Mah, A. L., Ford, D. L. Miller, J., Liang, J., Doong, H. and Monteiro, M I. (2004) Overexpression of ubiquilin decreases ubiquitination and degradation of presenilin proteins. J Alzheimers Dis, 6, 79-92.
23. Ko, H. 5., Uehara, T., Tsuruma, K. and Nomura, Y. (2004) Ubiquilin interacts with ubiquitylated proteins and proteasome through its ubiquitin-associated and ubiquitin-like domains. FEBS Lett, 566, 110-4.
24. Kleijnen, M. F., Shih, A. H., Zhou, P., Kumar, S., Soccio, R. E., Kedersha, N. L., Gill, G. and Rowley, P. M. (2000) The hPLIC proteins may provide a link between the ubiquitination machinery and the proteasome. Mol Cell, 6, 409-19.
25. Kleijnen, M. F., Alarcon, R. M. and Rowley, P. M. (2003) The ubiquitin-associated domain of hPLIC-2 interacts with the proteasome. Mol Bioi Cell, 14, 3868-75.
26. Wu, S., Mikhailov, A., Kallo-Hosein, H., Hara, K., Yonezawa, K. and Avruch, J. (2002) Characterization of ubiquilin 1, an rnTOR-interacting protein, Biochim Biophys Acta, 1542, 4 1-56.
27. Kaye, F. J., Modi, S., Ivanovska, I., Koonin, E. V., Thress, K., Kubo, A., Kornbluth, S. and Rose, M. D. (2000) A family of ubiquitin-like proteins binds the ATPase domain of Hsp7O-like Stch. FEBS Lett, 467, 348-55.
28. Ko, H. S., Uehara, T. and Nomura, Y. (2002) Role of ubiquilin associated with proteindisuffide isomerase in the endoplasmic reticulum in stress-induced apoptotic cell death. J Biol Chem, 277, 353 86-92.
29. Doi, H., Mitsui, K., Kurosawa, M., Machida, Y., Kuroiwa, Y. and Nukina, N. (2004) Identification of ubiquitin-interacting proteins in purified polyglutamine aggregates. FEBS Lett, 571, 171-6.
30. Mali, A. L., Perry, G., Smith, M. A. and Monteiro, M. J. (2000) Identification of ubiquilin, a novel presenilin interactor that increases presenilin protein accumulation. J Cell Biol, 151, 847-62.
31. Ford, D L. and Monteiro, M. J. (2006) Dimerization of ubiquilin is dependent upon the central region of the protein: evidence that the monomer, but not the dimer, is involved in binding presenilins. Biochem J, 399, 397-404.
32. Thomas, A N., Hen, L., Spoelgen, R., Hiltunen, M., Jones, P B., Tanzi, R. E., Hyman, B. T. and Berezovska, 0. (2006) Interaction between presenilin 1 and ubiquilin 1 as detected by fluorescence lifetime imaging microscopy and a high-throughput fluorescent plate reader. J Biol Chem, 281, 26400-7.
33. Bertram, L., Hiltunen, M., Parkinson, M., Ingeisson. M., Lange, C., Ramasarny, K., Mullin, K., Menon, R., Sampson, A. J., Hsiao, M. Y. et al. (2005) Family-based association between Alzheimer's disease and variants in UBQLN 1. N Eng J Med. 352, 884-94.
34. Brouwers, N., Sleegers, K., Engelborghs, S., Bogaerts, V., van Duijn, C. M., De Deyn, P. P., Van Broeckhoven, C. and Dermaut, B. (2006) The UBQLN1 polymorphism, UBQ8i, at 9q22 is not associated with Alzheimer's disease with onset before 70 years. Neurosci Lets, 392, 72-4.
35. Kamboh, M I., Minster, R. L., Feingold, F. and DeKosky, S. T. (2006) Genetic association of ubiquilin with Alzheimer's disease and related quantitative measures. Mol Psychiatry, 11, 273-9.
36. Slifer, M A., Martin, E. R., Bronson, P. G., Browning-Large, C., Doraiswamy, P. M., Welsh-Bohmer, K. A., Gilbert, J. R., Haines, J. L. and Pericak-Vance, M. A. (2006) Lack of association between UBQLN1 and Alzheimer disease. Am I Med Genet B Neuropsychiatr Genet, 141, 208-13.
37. Smemo, S., Nowotny, P., Hinrichs, A L., Kauwe, J. S., Cherny, S., Erickson, K., Myers, A. J., Kaleem, M., Marlowe, L., Gibson, A M. et al. (2006) Ubiquilin 1 polymorphisms are not associated with late-onset Alzheimers disease. Ann Neutvi, 59, 21-6.
38. Perry, R. T., Wiener, H., Hand!, L. E., Blacker, D., Tanzi, R E., Bertram, L., Bassett, S. S. and Go, R. C. (2006) Follow-up mapping supports the evidence for linkage in the candidate region at 9q22 in the NIMH Alzheimers disease Genetics Initiative cohort. Am J Med Genet B Neumpsychiatr Genet.

1. A method for decreasing cell death in a cell exhibiting aggregation of polyalanine-containing proteins, the method comprising:
introducing an expression vector to a host cell comprising a nucleotide sequence encoding ubiquilin; and
maintaining the transformed host cell under biological conditions sufficient for expression and accumulation of the ubiquilin in the host cell, to decrease cell death.

2. The method of claim 1, wherein the expression vector comprises a nucleotide sequence that encodes polypeptides comprising the amino acid residue of ubiquilin, or variants having at least 90% homology and having the same functional activity of ubiquilin, or fragments thereof.

3. The method according to claim 1, wherein the nucleotide sequence is SEQ ID NOs: 1, 3, 5, 9, 11, or 13.

4. A method for determining the effectiveness of ubiquilin in reducing polyalanine expansion in a host cell, the method comprising:
introducing an expression vector to a host cell comprising a nucleotide sequence encoding ubiquilin; maintaining the transformed host cell under biological conditions sufficient for expression and accumulation of the ubiquilin in the host cell; and
measuring and comparing the level of cell death in the host cells expressing ubiquilin relative to a host cell not expressing an increased level of ubiquilin,
wherein a decrease in the level of cell death in the host cells expressing ubiquilin relative to a host cell not expressing increased levels of ubiquilin demonstrates the effectiveness of ubiquilin in reducing polyalanine expansion in the host cell.

5. A method of treatment of disease associated with expanded polyalanine and polyglutamine proteins, or disease associated with misfolding and aggregation of unrelated proteins, or a neurological disorder, comprising administration, to a subject afflicted therewith, of an expression vector encoding for ubiquilin protein or variant thereof having deletions or substitutions, wherein the variant, when expressed maintains functional activity of ubiquilin.

6. The method of claim 5, wherein the neurological disorder comprises Huntington's disease.

7. A method of reducing polyalanine protein aggregates and cell death, comprising overexpressing ubiquilin in a cellular locus susceptible to such aggregates and cell death.

8. A method of clearing misfolded protein aggregates from accumulating in cells in a cellular locus, comprising overexpressing ubiquilin in said cellular locus.

9. A method of treating disease associated with expanded polyalanine proteins in a cellular locus, comprising overexpressing ubiquilin in said cellular locus.

10. A method of preventing toxicity induced by expanded polyalanine proteins in a cellular locus, comprising overexpressing ubiquilin in said cellular locus.

11. The method of claim 4, wherein the polyalanine expansion comprises an expansion of at least two residues above a threshold of 20 residues.

12. The method of claim 4, wherein the expression vector comprises a nucleotide sequence that encodes polypeptides comprising the amino acid residue of ubiquilin, or variants having at least 90% homology and having the same functional activity of ubiquilin, or fragments thereof.

13. The method of claim 4, wherein the nucleotide sequence is SEQ ID NOs: 1, 3, 5, 9, 11, or 13.

14. The method of claim 5, wherein the polyalanine expansion comprises an expansion of at least two residues above a threshold of 20 residues.

15. The method of claim 5, wherein the expression vector comprises a nucleotide sequence that encodes polypeptides comprising the amino acid residue of ubiquilin, or variants having at least 90% homology and having the same functional activity of ubiquilin, or fragments thereof.

16. The method of claim 5, wherein the nucleotide sequence is SEQ ID NOs: 1, 3, 5, 9, 11, or 13.

17. The method of claim 7, wherein the overexpressing of ubiquilin comprises administration of an expression vector comprising a nucleotide sequence that encodes polypeptides comprising the amino acid residue of ubiquilin, or variants having at least 90% homology and having the same functional activity of ubiquilin, or fragments thereof.

18. The method of claim 8, wherein the overexpressing of ubiquilin comprises administration of an expression vector comprising a nucleotide sequence that encodes polypeptides comprising the amino acid residue of ubiquilin, or variants having at least 90% homology and having the same functional activity of ubiquilin, or fragments thereof.

19. The method of claim 9, wherein the overexpressing of ubiquilin comprises administration of an expression vector comprising a nucleotide sequence that encodes polypeptides comprising the amino acid residue of ubiquilin, or variants having at least 90% homology and having the same functional activity of ubiquilin, or fragments thereof.

20. The method of claim 10, wherein the overexpressing of ubiquilin comprises administration of an expression vector comprising a nucleotide sequence that encodes polypeptides comprising the amino acid residue of ubiquilin, or variants having at least 90% homology and having the same functional activity of ubiquilin, or fragments thereof.


SEQUENCE LISTING (18 sequences)

SEQ ID NO: 1 - DNA, 1770 bp, homo sapiens
atggccgagagtggtgaaagcggcggtcctccgggctcccaggatagcgccgccggagccgaaggtgctggcgcccccgcggccgctgcctccgcggagcccaaaatcatgaaagtcaccgtgaagaccccgaaggaaaaggaggaattcgccgtgcccgagaatagctccgtccagcagtttaaggaagaaatctctaaacgttttaaatcacatactgaccaacttgtgttgatatttgctggaaaaattttgaaagatcaagataccttgagtcagcatggaattcatgatggacttactgttcaccttgtcattaaaacacaaaacaggcctcaggatcattcagctcagcaaacaaatacagctggaagcaatgttactacatcatcaactcctaatagtaactctacatctggttctgctactagcaacccttttggtttaggtggccttgggggacttgcaggtctgagtagcttgggtttgaatactaccaacttctctgaactacagagtcagatgcagcgacaacttttgtctaaccctgaaatgatggtccagatcatggaaaatccctttgttcagagcatgctctcaaatcctgacctgatgagacagttaattatggccaatccacaaatgcagcagttgatacagagaaatccagaaattagtcatatgttgaataatccagatataatgagacaaacgttggaacttgccaggaatccagcaatgatgcaggagatgatgaggaaccaggaccgagctttgagcaacctagaaagcatcccagggggatataatgctttaaggcgcatgtacacagatattcaggaaccaatgctgagtgctgcacaagagcagtttggtggtaatccatttgcttccttggtgagcaatacatcctctggtgaaggtagtcaaccttcccgtacagaaaatagagatccactacccaatccatgggctccacagacttcccagagttcatcagcttccagcggcactgccagcactgtgggtggcactactggtagtactgccagtggcacttctgggcagagtactactgcgccaaatttggtgcctggagtaggagctagtatgttcaacacaccaggaatgcagagcttgttgcaacaaataactgaaaacccacaacttatgcaaaacatgttgtctgccccctacatgagaagcatgatgcagtcactaagccagaatcctgaccttgctgcacagatgatgctgaataatcccctatttgctggaaatcctcagcttcaagaacaaatgagacaacagctcccaactttcctccaacaaatgcagaatcctgatacactatcagcaatgtcaaaccctagagcaatgcaggccttgttacagattcagcagggtttacagacattagcaacggaagccccgggcctcatcccagggtttactcctggcttgggggcattaggaagcactggaggctcttcgggaactaatggatctaacgccacacctagtgaaaacacaagtcccacagcaggaaccactgaacctggacatcagcagtttattcagcagatgctgcaggctcttgctggagtaaatcctcagctacagaatccagaagtcagatttcagcaacaactggaacaactcagtgcaatgggatttttgaaccgtgaagcaaacttgcaagctctaatagcaacaggaggtgatatcaatgcagctattgaaaggttactgggctcccagccatcatag

SEQ ID NO: 2 - protein, 589 aa, homo sapiens
MAESGESGGPPGSQDSAAGAEGAGAPAAAASAEPKIMKVTVKTPKEKEEFAVPENSSVQQFKEEISKRFKSHTDQLVLIFAGKILKDQDTLSQHGIHDGLTVHLVIKTQNRPQDHSAQQTNTAGSNVTTSSTPNSNSTSGSATSNPFGLGGLGGLAGLSSLGLNTTNFSELQSQMQRQLLSNPEMMVQIMENPFVQSMLSNPDLMRQLIMANPQMQQLIQRNPEISHMLNNPDIMRQTLELARNPAMMQEMMRNQDRALSNLESIPGGYNALRRMYTDIQEPMLSAAQEQFGGNPFASLVSNTSSGEGSQPSRTENRDPLPNPWAPQTSQSSSASSGTASTVGGTTGSTASGTSGQSTTAPNLVPGVGASMFNTPGMQSLLQQITENPQLMQNMLSAPYMRSMMQSLSQNPDLAAQMMLNNPLFAGNPQLQEQMRQQLPTFLQQMQNPDTLSAMSNPRAMQALLQIQQGLQTLATEAPGLIPGFTPGLGALGSTGGSSGTNGSNATPSENTSPTAGTTEPGHQQFIQQMLQALAGVNPQLQNPEVRFQQQLEQLSAMGFLNREANLQALIATGGDINAAIERLLGSQPS

SEQ ID NO: 3 - DNA, 2974 bp, Homo sapiens
ggaggaagcggtggctgctgcggatgtcggtgtgagcgagcggcgcctgaacacacggcggctgccgagcgcctgacccgggcctgcgccagagcctgcaccgagctccggggccccacacccgctacggtggccctgcgcccgttgctactgaggcggcgtgctctgcattcttcgctgtccaggcctgccggctctggtgtctgctggctcctccttgctcgcctgctccctcctgcttgcctgagtcaccgccgccgccgccgccacagccatggccgagagtggtgaaagcggcggtcctccgggctcccaggatagcgccgccggagccgaaggtgctggcgcccccgcggccgctgcctccgcggagcccaaaatcatgaaagtcaccgtgaagaccccgaaggaaaaggaggaattcgccgtgcccgagaatagctccgtccagcagtttaaggaagaaatctctaaacgttttaaatcacatactgaccaacttgtgttgatatttgctggaaaaattttgaaagatcaagataccttgagtcagcatggaattcatgatggacttactgttcaccttgtcattaaaacacaaaacaggcctcaggatcattcagctcagcaaacaaatacagctggaagcaatgttactacatcatcaactcctaatagtaactctacatctggttctgctactagcaacccttttggtttaggtggccttgggggacttgcaggtctgagtagcttgggtttgaatactaccaacttctctgaactacagagtcagatgcagcgacaacttttgtctaaccctgaaatgatggtccagatcatggaaaatccctttgttcagagcatgctctcaaatcctgacctgatgagacagttaattatggccaatccacaaatgcagcagttgatacagagaaatccagaaattagtcatatgttgaataatccagatataatgagacaaacgttggaacttgccaggaatccagcaatgatgcaggagatgatgaggaaccaggaccgagctttgagcaacctagaaagcatcccagggggatataatgctttaaggcgcatgtacacagatattcaggaaccaatgctgagtgctgcacaagagcagtttggtggtaatccatttgcttccttggtgagcaatacatcctctggtgaaggtagtcaaccttcccgtacagaaaatagagatccactacccaatccatgggctccacagacttcccagagttcatcagcttccagcggcactgccagcactgtgggtggcactactggtagtactgccagtggcacttctgggcagagtactactgcgccaaatttggtgcctggagtaggagctagtatgttcaacacaccaggaatgcagagcttgttgcaacaaataactgaaaacccacaactgatgcaaaacatgttgtctgccccctacatgagaagcatgatgcagtcactaagccagaatcctgaccttgctgcacagatgatgctgaataatcccctatttgctggaaatcctcagcttcaagaacaaatgagacaacagctcccaactttcctccaacaaatgcagaatcctgatacactatcagcaatgtcaaaccctagagcaatgcaggccttgttacagattcagcagggtttacagacattagcaacggaagccccgggcctcatcccagggtttactcctggcttgggggcattaggaagcactggaggctcttcgggaactaatggatctaacgccacacctagtgaaaacacaagtcccacagcaggaaccactgaacctggacatcagcagtttattcagcagatgctgcaggctcttgctggagtaaatcctcagctacagaatccagaagtcagatttcagcaacaactggaacaactcagtgcaatgggatttttgaaccgtgaagcaaacttgcaagctctaatagcaacaggaggtgatatcaatgcagctattgaaaggttactgggctcccagccatcatagcagcatttctgtatcttgaaaaaatgtaatttatttttgataacggctcttaaactttaaaatacctgctttatttcattttgactcttggaattctgtgctgttataaacaaacccaatatgatgcattttaaggtggagtacagtaagatgtgtgggtttttctgtatttttcttttctggaacagtgggaattaaggctactgcatgcatcacttctgcatttattgtaattttttaaaaacatcaccttttatagttgggtgaccagattttgtcctgcatctgtccagtttatttgctttttaaacattagcctatggtagtaatttatgtagaataaaagcattaaaaagaagcaaatcatttgcactctataatttgtggtacagtattgcttattgtgactttggcatgcatttttgcaaacaatgctgtaagatttatactactgataattttgttttatttgtatacaatatagagtatgcacatttgggactgcatttctggaaacatactgcaataggctctctgagcaaaacacctgtaactaaaaaagtgaagataagaaaatactcttaaagctgagtatttcctaattgtatagaatcttacagcatctttgacaaacatctcccagcaaaagtgccggttagtcaggtttgttgaaaatacagtagaaaagctgattctggttatctctttaaggacaattaattgtacagacacataatgtaacattgtctcaacattcattcacagattgactgtaaattaccttaatctttgtgcagactgaaggaacactgtagtataccccaaagtgcatttgcctaggacttctcagcttctcccataggtagtttaacaggcattaaaatttgtaattgaaatgttgctttcactcaaaaaaaaaaaaaaaaaaaa

SEQ ID NO: 4 - protein, 589 aa, Homo sapiens; identical to SEQ ID NO: 2

SEQ ID NO: 5 - DNA, 1770 bp, Homo sapiens; identical to SEQ ID NO: 1

SEQ ID NO: 6 - protein, 589 aa, Homo sapiens; identical to SEQ ID NO: 2

SEQ ID NO: 7 - DNA, 2354 bp, Homo sapiens
ggaggaagcggtggctgctgcggatgtcggtgtgagcgagcggcgcctgaacacacggcggctgccgagcgcctgacccgggcctgcgccagagcctgcaccgagctccggggccccacacccgctacggtggccctgcgtccaggcctgccggctctggtgcctgagtcaccgccgccgtcctccgggctcccaggatatgcctccgcggagcccaaaaattcgccgtgcccgagaatataaatcacatactgaccaactaccttgagtcagcatggaaaaacaggcctcaggatcattatcatcaactcctaatagtaaggtggccttgggggacttgtgaactacagagtcagatgccatggaaaatccctttgttctatggccaatccacaaatgcgaataatccagatataatgaggagatgatgaggaaccaggtaatgctttaaggcgcatgtgcagtttggtggtaatccattcaaccttcccgtacagaaaccagagttcatcagcttccatgccagtggcacttctgggctagtatgttcaacacaccagactgatgcaaaacatgttgtgaatcctgaccttgctgcactagagcaatgcaggccttgtcccgggcctcatcccagggtttcgggaactaatggatctacactgaacctggacatcagctcctcagctacagaatccaggggatttttgaaccgtgaagtgcagctattgaaaggttacaaaatgtaatttatttttgattgactcttggaattctgtggtacagtaagatgtgtgggtctactgcatgcatcacttcttgggtgaccagattttgtcctggtagtaatttatgtagaaaaaaaaaaaaaaaacccgttgctactgaggcggcgtgctctgcattcttcgctgtgtctgctggctcctccttgctcgcctgctccctcctgctccgccgccacagccatggccgagagtggtgaaagcggcgggcgccgccggagccgaaggtgctggcgcccccgcggccgctcatgaaagtcaccgtgaagaccccgaaggaaaaggaggagctccgtccagcagtttaaggaagaaatctctaaacgtttttgtgttgatatttgctggaaaaattttgaaagatcaagattcatgatggacttactgttcaccttgtcattaaaacacacagctcagcaaacaaatacagctggaagcaatgttactacactctacatctggttctgctactagcaacccttttggtttcaggtctgagtagcttgggtttgaatactaccaacttctcagcgacaacttttgtctaaccctgaaatgatggtccagatagagcatgctctcaaatcctgacctgatgagacagttaatagcagttgatacagagaaatccagaaattagtcatatgttgacaaacgttggaacttgccaggaatccagcaatgatgcaaccgagctttgagcaacctagaaagcatcccagggggataacacagatattcaggaaccaatgctgagtgctgcacaagattgcttccttggtgagcaatacatcctctggtgaaggtagatagagatccactacccaatccatgggctccacagacttcgcggcactgccagcactgtgggtggcactactggtagtacagagtactactgcgccaaatttggtgcctggagtaggagcgaatgcagagcttgttgcaacaaataactgaaaacccacactgccccctacatgagaagcatgatgcagtcactaagccaagatgcagaatcctgatacactatcagcaatgtcaaaccctacagattcagcagggtttacagacattagcaacggaagcttactcctggcttgggggcattaggaagcactggaggctcacgccacacctagtgaaaacacaagtcccacagcaggaacagtttattcagcagatgctgcaggctcttgctggagtaaaaagtcagatttcagcaacaactggaacaactcagtgcaatcaaacttgcaagctctaatagcaacaggaggtgatatcaatgggctcccagccatcatagcagcatttctgtatcttgaataacggctcttaaactttaaaatacctgctttatttcattctgttataaacaaacccaatatgatgcattttaaggtggattttctgtatttttcttttctggaacagtgggaattaagggcatttattgtaattttttaaaaacatcaccttttatagttgcatctgtccagtttatttgctttttaaacattagcctataaaagcattaaaaaaaaaaaaaaaaaaaaaaaaaaaaaa

SEQ ID NO: 8 - protein, 561 aa, Homo sapiens
MAESGESGGPPGSQDSAAGAEGAGAPAAAASAEPKIMKVTVKTPKEKEEFAVPENSSVQQFKEEISKRFKSHTDQLVLIFAGKILKDQDTLSQHGIHDGLTVHLVIKTQNRPQDHSAQQTNTAGSNVTTSSTPNSNSTSGSATSNPFGLGGLGGLAGLSSLGLNTTNFSELQSQMQRQLLSNPEMMVQIMENPFVQSMLSNPDLMRQLIMANPQMQQLIQRNPEISHMLNNPDIMRQTLELARNPAMMQEMMRNQDRALSNLESIPGGYNALRRMYTDIQEPMLSAAQEQFGGNPFASLVSNTSSGEGSQPSRTENRDPLPNPWAPQTSQSSSASSGTASTVGGTTGSTASGTSGQSTTAPNLVPGVGASMFNTPGMQSLLQQITENPQLMQNMLSAPYMRSMMQSLSQNPDLAAQMQNPDTLSAMSNPRAMQALLQIQQGLQTLATEAPGLIPGFTPGLGALGSTGGSSGTNGSNATPSENTSPTAGTTEPGHQQFIQQMLQALAGVNPQLQNPEVRFQQQLEQLSAMGFLNREANLQALIATGGDINAAIERLLGSQPS

SEQ ID NO: 9 - DNA, 3952 bp, Homo sapiens
gggatgactcggcgacgccgccaggcgcgtagggacgtgggggaaggggcagggaggagggcgagcggcgcctgaacacacggcggctgcctgcaccgagctccggggccccacacccgcgcggcgtgctctgcattcttcgctgtccagccttgctcgcctgctccctcctgcttgccttggccgagagtggtgaaagcggcggtcctctacccggcgtgctccgcgcggcgccagcgaaagcggtggctgctgcggatgtcggtgtgacgagcgcctgacccgggcctgcgccagagctacggtggccctgcgcccgttgctactgaggcctgccggctctggtgtctgctggctcctgagtcaccgccgccgccgccgccacagccacgggctcccaggatagcgccgccggagccgaaggtgctggcgcccccgcggccgctgcctccgcggagcccaaaatcatgaaagtcaccgtgaagaccccgaaggaaaaggaggaattcgccgtgcccgagaatagctccgtccagcagtttaaggaagaaatctctaaacgttttaaatcacatactgaccaacttgtgttgatatttgctggaaaaattttgaaagatcaagataccttgagtcagcatggaattcatgatggacttactgttcaccttgtcattaaaacacaaaacaggcctcaggatcattcagctcagcaaacaaatacagctggaagcaatgttactacatcatcaactcctaatagtaactctacatctggttctgctactagcaacccttttggtttaggtggccttgggggacttgcaggtctgagtagcttgggtttgaatactaccaacttctctgaactacagagtcagatgcagcgacaacttttgtctaaccctgaaatgatggtccagatcatggaaaatccctttgttcagagcatgctctcaaatcctgacctgatgagacagttaattatggccaatccacaaatgcagcagttgatacagagaaatccagaaattagtcatatgttgaataatccagatataatgagacaaacgttggaacttgccaggaatccagcaatgatgcaggagatgatgaggaaccaggaccgagctttgagcaacctagaaagcatcccagggggatataatgctttaaggcgcatgtacacagatattcaggaaccaatgctgagtgctgcacaagagcagtttggtggtaatccatttgcttccttggtgagcaatacatcctctggtgaaggtagtcaaccttcccgtacagaaaatagagatccactacccaatccatgggctccacagacttcccagagttcatcagcttccagcggcactgccagcactgtgggtggcactactggtagtactgccacaaatttggtgcctggagtaggagctagtatgcaacaaataactgaaaacccacaactgagaagcatgatgcagtcactaagccagaatcatcccctatttgctggaaatcctcagcttctcctccaacaaatgcagaatcctgatacacaggccttgttacagattcagcagggtttactcccagggtttactcctggcttgggggcatatggatctaacgccacacctagtgaaaacagacatcagcagtttattcagcagatgctgcagaatccagaagtcagatttcagcaacaacaccgtgaagcaaacttgcaagctctaatagaaaggttactgggctcccagccatcatagctatttttgataacggctcttaaactttaaaaattctgtgctgttataaacaaacccaatatgtgtgggtttttctgtatttttcttttctatcacttctgcatttattgtaattttttaaattttgtcctgcatctgtccagtttatttgtatgtagaataaaagcattaaaaagaagcagtattgcttattgtgactttggcatgcattctgataattttgttttatttgtatacaataggaaacatactgcaataggctctctgagcagaaaatactcttaaagctgagtatttcctaaacatctcccagcaaaagtgccggttagtcattctggttatctctttaaggacaattaatacattcattcacagattgactgtaaattacgtagtataccccaaagtgcatttgcctaggcaggcattaaaatttgtaattgaaatgttggttatttttaatcgccataaaaaaatagaacacaggcaatacacaaatttaaaatgagttagttctattaagaaatagttaaatattgtgggtgggggggtggggcagcggaatctgtccaccaagattctgttaggatttctgtgcatagaaaaacaaagagccgttttaatgatgttggtggcacttctgggcagagtactactgcgctgttcaacacaccaggaatgcagagcttgttgcaaaacatgttgtctgccccctacatgactgaccttgctgcacagatgatgctgaataaagaacaaatgagacaacagctcccaactttatcagcaatgtcaaaccctagagcaatgcagacattagcaacggaagccccgggcctcataggaagcactggaggctcttcgggaactacaagtcccacagcaggaaccactgaacctgaggctcttgctggagtaaatcctcagctactggaacaactcagtgcaatgggatttttgacaacaggaggtgatatcaatgcagctattgagcatttctgtatcttgaaaaaatgtaattatacctgctttatttcattttgactcttggtgatgcattttaaggtggagtacagtaagaggaacagtgggaattaaggctactgcatgcaaacatcaccttttatagttgggttaccagctttttaaacattagcctatggtagtaattaatcatttgcactctataatttgtggtacatttgcaaacaatgctgtaagatttatactatagagtatgcacatttgggactgcatttctaaacacctgtaactaaaaaagtgaagataaattgtatagaatcttacagcatctttgacaaggtttgttgaaaatacagtagaaaagctgtgtacagacacataatgtaacattgtctcacttaatctttgtgcagactgaaggaacactacttctcagcttctcccataggtagtttaactttcactgaaaaagtgtcttgatgtttcactatcttttgggtttatctgttttctcatggtgagccaattgtttctgaagtgttttggtcttttcagagcctcagagaaagggggacggtggatggggccagcttaaataatactggcatagtgtagtaaagaagtatcattcaggggtagtacatttggctgttttatagcctttttcttccctcccccaaagaattctgtttgcctaactcccaaactgttggggtggtacattcctttaggaccaattaaaacataattgagggtcagtgatacatttggctgactctggttcagtattctcttaggtgattatattctctcatgtacagttacaggaaattaaaatgttaaagtaacctaaaatgaattcagaccaataaaatcaagggaaatacaagttgattgcattacttctgtatgttgcttgctattaaaaaggttaagaggccaggttacccaccagtccttgcactgttctgacactttccccaggaggaaaacaagtacaaaggttacggtggaggcataagtagaagagattgttaagaagggtattcatgtgtctttgctctttctgctttatgcctcagtttggtttaaaaacttctgtactggcaaatggtggtattcagtgtgggatagtgtcataactaatttgacaatttattaatcataaaataacaataaatctctagcttttacacttg

SEQ ID NO: 10 - protein, 589 aa, Homo sapiens; identical to SEQ ID NO: 2

SEQ ID NO: 11 - DNA, 3018 bp, Homo sapiens
atggctacagagagtgcactcatcaaagttcacgtgaaatcaccgtcgaacaagtatgactaccgatgtctctcacgtgagtagtttcaagtgcactttagtggcagcttgttcatactggttgagattgctgcggatgcatcagtttcagaactaaaagacaaggttcttgtgttcgttcaactctaacgacgcctacgtagtcaaagtcttgattttctgttccaagaacacaagcaaccaactgcgaacaaagaacaagtttgtataatttacaccggaaaaattctgaaggatgaaggttgacgcttgtttcttgttcaaacatattaaatgtggcctttttaagacttcctacttgaaactctcacacagcacaaaatcgctgatggtcacaccgtccacttggttattagaaatctttgagagtgtgtcgtgttttagcgactaccagtgtggcaggtgaaccaataatctttacaagcccgtccaacaccagcgccggctgctgcaacaccaacagcttcatctgcaccaagtgttcgggcaggttgtggtcgcggccgacgacgttgtggttgtcgaagtagacgtggttcatccaatccaacaccttcttcacaaccgaatcctaccaataatccatttgcagcaatgggaaggttaggttgtggaagaagtgttggcttaggatggttattaggtaaacgtcgttaccctggaatgggatcacctgctgatattttaaacaatccagatgctatgcgttcagttatggatccttaccctagtggacgactataaaatttgttaggtctacgatacgcaagtcaatacctaaatccaattacacaacaacttctaggaaatccagagtttatgagaacaattattcaatccttaggttaatgtgttgttgaagatcctttaggtctcaaatactcttgttaataagttaggaacccacaattccaagcattgattgagagaaatccagaagttggacacattctcaacgatttgggtgttaaggttcgtaactaactctctttaggtcttcaacctgtgtaagagttgctaccgaatgtaatgcgtcaaactatggagatgattcgtaatccaaatatgttccaagaaatgggcttacattacgcagtttgatacctctactaagcattaggtttatacaaggttctttacatgcggaatcatgatcaagctattaggaatcttcagggaattcccggaggagaagctgcttacgccttagtactagttcgataatccttagaagtcccttaagggcctcctcttcgacgacttgaacggttgtacaatgatgtacaggagcccttgctcaatagtgcaacaaattcgctcgaacttgccaacatgttactacatgtcctcgggaacgagttatcacgttgtttaagcgagagtggaaatccatttgcttctctaagaggtgatcagagcagtgagccacgtgtcgatcgttcacctttaggtaaacgaagagattctccactagtctcgtcactcggtgcacagctagcagctggacaagaaaataatgaagctctgccaaatccatgggcttcaaacgccaatcaagctcgacctgttcttttattacttcgagacggtttaggtacccgaagtttgcggttagttcgaaccaataatcaatcgaataatcgttctgctgactttaattcattgcttgattcacctggctggttattagttagcttattagcaagacgactgaaattaagtaacgaactaagtggaccgatcagctccctaatggagcagatgatgtccaatccaagtatgcaggccagtatgttcagctagtcgagggattacctcgtctactacaggttaggttcatacgtccggtcatacaagtcgccagaagtcatcaattcaattcgtcaaaatatgtcaaacaatcctggactcattgattccggtcttcagtagttaagttaagcagttttatacagtttgttaggacctgagtaactaaggattgttggacaaattccatcggctcgtgataacccacagatttccgaaggaattcgtagataacaacctgtttaaggtagccgagcactattgggtgtctaaaggcttccttaagcatctagttttccacaaatgctcaacatgatgtctgatccatctgtgatggaggcaatgagaaactcaaaaggtgtttacgagttgtactacagactaggtagacactacctccgttactctttgccacgggtcagtgaagcattccgtcaaattcaagagggattttcaactcttcgcagagaaggtgcccagtcacttcgtaaggcagtttaagttctccctaaaagttgagaagcgtctcttgcacctcaacttttgaaccttttccaagctggagcaatgggaggtggagcattcggttctcgtggagttgaaaacttggaaaaggttcgacctcgttaccctccacctcgtaagccaagagatgccaatgcatcttctgctggagcaaactctgcaaacggacttgccgatttattcaatctacggttacgtagaagacgacctcgtttgagacgtttgcctgaacggctaaataagttatcaatgaatatgggaggaggaagaccctcatcgactgctgcaccagtgaatccggagcaaagttacttataccctcctccttctgggagtagctgacgacgtggtcacttaggcctcgttacctatgcatcacaactcgaacaacttcaatcgatgggcttctcggatcgtgccagaaattggatacgtagtgttgagcttgttgaagttagctacccgaagagcctagcacggtctttagtggcagcgctgaccgcaacgttcggagatctcaacgcggctgtcgaacgtcttctcaaccaccgtcgcgactggcgttgcaagcctctagagttgcgccgacagcttgcagaagagttgtctccatagagaggtatc

SEQ ID NO: 12 - protein, 502 aa, Homo sapiens
MATESALIKVHVKSPSNKYDVEIAADASVSELKDKVLVFVPTANKEQVCIIYTGKILKDEETLTQHKIADGHTVHLVIRNQARPTPAPAAATPTASSAPSSNPTPSSQPNPTNNPFAAMGGMGSPADILNNPDAMRSVMDNPITQQLLGNPEFMRTIIQSNPQFQALIERNPEVGHILNDPNVMRQTMEMIRNPNMFQEMMRNHDQAIRNLQGIPGGEAALERLYNDVQEPLLNSATNSLSGNPFASLRGDQSSEPRVDRAGQENNEALPNPWASNANQATNNQSNNRSADFNSLLDSPGISSLMEQMMSNPSMQASMFSPEVINSIRQNMSNNPGLIDSIVGQIPSARDNPQISEGIRRSFPQMLNMMSDPSVMEAMRNPRVSEAFRQIQEGFSTLRREAPQLLNLFQAGAMGGGAFGSDANASSAGANSANGLADLFNSMNMGGGRPSSTAAPVNPEQTYASQLEQLQSMGFSDRARNVAALTATFGDLNAAVERLLNSP

SEQ ID NO: 13 - DNA, 3324 bp, Homo sapiens
cggaggaggcccagagaccggagcgcggagacctcagccagcggcctacgcccaggcctttctccaccggaggaccagggaaccgcagtcttcatcacagaggtaccgtgctccgcgctccccgcctgacccggcccagcccgctgcggcggtgcctccttccttcctccttccctcgcgctctctctttcgcccgcccgcgccttccctgcccgcctgcgtcaccgcggccgccatggctgagaatggcgagagcagcggccccccgcgcccctcccgcggccctgctgcggcccaaggctcggctgctgccccggctgagcctaaaatcatcaaagtcacggtgaagactcccaaagagaaagaggagttcgcggtgcccgagaacagctcggttcagcagtttaaggaagcgatttcgaaacgcttcaaatcccaaaccgatcagctagtgctgatttttgccggaaaaatcttaaaagatcaagataccttgatccagcatggcatccatgatgggctgactgttcaccttgtcatcaaaagccagaaccgacctcagggccagtccacgcagcctagcaatgccgcgggaactaacactacctcggcgtcgactcccaggagtaactccacacctatttccacaaatagcaacccgtttgggttggggagcctgggaggacttgcaggccttagcagcctgggcttgagctcgaccaacttctctgagctccagagccagatgcagcagcagcttatggccagccctgagatgatgatccaaataatggaaaatccctttgttcagagcatgctttcgaatcccgatctgatgaggcagctgattatggctaatccacagatgcagcaattgattcagagaaacccagaaatcagtcacctgctcaacaacccagacataatgaggcagacactcgaaattgccaggaatccagccatgatgcaagagatgatgagaaatcaagacctggctcttagcaatctagaaagcatcccaggtggctataatgctttacggcgcatgtacactgacattcaagagccgatgctgaatgccgcacaagagcagtttgggggtaatccatttgcctccgtggggagtagttcctcctctggggaaggtacgcagccttcccgcacagaaaatcgcgatccactacccaatccatgggcaccaccgccagctacccagagttctgcaactaccagcacgaccacaagcactggtagtgggtctggcaatagttccagcaatgctactgggaacaccgttgctgccgctaattatgtcgccagcatctttagtaccccaggcatgcagagcctgctgcaacagataactgaaaacccccagctgattcagaatatgctgtcggcgccctacatgagaagcatgatgcagtcgctgagccagaatccagatttggctgcacagatgatgctgaatagcccgctgtttactgcaaatcctcagctgcaggagcagatgcggccacagctcccagccttcctgcagcagatgcagaatccagacacactatcagccatgtcaaacccaagagcaatgcaggctttaatgcagatccagcaggggctacagacattagccactgaagcacctggcctgattccgagcttcactccaggtgtgggggtgggggtgctgggaaccgctataggccctgtaggcccagtcacccccataggccccataggccctatagtcccttttacccccataggccccattgggcccataggacccactggccctgcagccccccctggctccaccggctctggtggccccacggggcctactgtgtccagcgctgcacctagtgaaaccacgagtcctacatcagaatctggacccaaccagcagttcattcagcaaatggtgcaggccctggctggagcaaatgctccacagctgccgaatccagaagtcagatttcagcaacaactggaacagctcaacgcaatggggttcttaaaccgtgaagcaaacttgcaggccctaatagcaacaggaggcgacatcaatgcagccattgaaaggctgctgggctcccagccatcgtaatcacatttctgtacctggaaaaaaaatgtatcttatttttgataatggctcttaaatctttaaacacacacacaaaatcgttctttactttcattttgattcttttaaatctgtctagttgtaagtctaatatgatgcattttaagatggagtccctccctcctacttccctcactccctttctcctttgcttatttttcctaccttcccttcctcttgtctccccactccctccctctttgtttccttccttccttatttcctttagtttccttccttagccgttttgagtggtgggaatcaatgctgtttcactcaaaagtgttgcatgcaaacacttctctttattctgcatttattgtgatttttggaaacaggtatcaaccttcacagttgggtgaacaagtgttgtcctacagatgtccaatttatttgcatttttaaacattagcctatgatagtaatttaatgtagaatgaagatattaaaaacagaagcaaattatttgaagctctctaatttgtggtacgatattgcttattgtgactttggcatgtatttttgctagcaaaatgctgtaagatttataccattgatcttttttgctatatttgtatacagtacagtaagcacaattggcactgtacatctaaaaatattacagtagaatctgagtgtaatatgtgtaaccaaaatgagaaagaatacaagaaatgtttctggagctagttatgtctcacaattttgtagaatcttacagcatctttgataaacttctcagtgaaaatgttggctaggcaagttcagttaaaatatagtagaaatgtttatcctggtatctctaagtatacatttaattgtacagaaaatttacagtgtaacattgtgtcaacatttgcagattgactgtatatgaccttaatctttgtgcagcctgaaggatcagtgtagtaatgccaggaaagtgctttttacctaagacttccttctcagcttctcccataaagagaccctaatatgcattttgatttgtaattggaaatgtaactttcactgaaagtgtcatgtgatgtttgcattacttttaactgctatgtataaaggaaagtgtgtcttttgacttcatcagttatttctcttgtgcacagagaaaaatgcattaaaaatgactaaaaaaaataaaaaattaaaaaatgaaaaaaaaaaaaaaaaaa

SEQ ID NO: 14 - protein, 624 aa, Homo sapiens
MAENGESSGPPRPSRGPAAAQGSAAAPAEPKIIKVTVKTPKEKEEFAVPENSSVQQFKEAISKRFKSQTDQLVLIFAGKILKDQDTLIQHGIHDGLTVHLVIKSQNRPQGQSTQPSNAAGTNTTSASTPRSNSTPISTNSNPFGLGSLGGLAGLSSLGLSSTNFSELQSQMQQQLMASPEMMIQIMENPFVQSMLSNPDLMRQLIMANPQMQQLIQRNPEISHLLNNPDIMRQTLEIARNPAMMQEMMRNQDLALSNLESIPGGYNALRRMYTDIQEPMLNAAQEQFGGNPFASVGSSSSSGEGTQPSRTENRDPLPNPWAPPPATQSSATTSTTTSTGSGSGNSSSNATGNTVAAANYVASIFSTPGMQSLLQQITENPQLIQNMLSAPYMRSMMQSLSQNPDLAAQMMLNSPLFTANPQLQEQMRPQLPAFLQQMQNPDTLSAMSNPRAMQALMQIQQGLQTLATEAPGLIPSFTPGVGVGVLGTAIGPVGPVTPIGPIGPIVPFTPIGPIGPIGPTGPAAPPGSTGSGGPTGPTVSSAAPSETTSPTSESGPNQQFIQQMVQALAGANAPQLPNPEVRFQQQLEQLNAMGFLNREANLQALIATGGDINAAIERLLGSQPS

SEQ ID NO: 15 - DNA, 2347 bp, Homo sapiens
gggaggtttggagccctgcataaagagaaggacgggaccacagctgactgctgtgtccccacagatctgggcctcctgctgccaccatggccaaaggtggagaagccctgccacagggcagcccagcaccagtccaggatccccacctcatcaaggtgacagtgaagacgcccaaagacaaggaggatttctcagttacagacacatgcactatccagcagctgaaggaagagatatctcagcgctttaaggcccaccccgatcagcttgttctaatctttgctggcaaaatcctcaaggatcctgactcactggcacagtgtggagtgcgagatggcctcactgtccacctggtcatcaagaggcagcaccgtgccatgggcaatgagtgcccagctgcctctgtccctacccagggcccaagtcctggatcactccctcagccaagctccatttacccagcagatgggccccctgcctttagcttaggtctcctcacaggcctcagtaggctgggcttggcctatcgtggcttccctgaccagccaagctccctgatgcggcagcatgtgtctgtgcctgagtttgtgactcagctcattgatgaccccttcatcccgggtctgctgtccaacacaggcctagtacgccagctggttcttgacaacccccatatgcagcagctgatccagcacaaccctgagattgggcatattcttaacaacccggaaattatgcggcagacactggagtttttacgtaaccctgccatgatgcaggagatgatacgtagccaggaccgggtgctcagtaacttggagagcattcctggtggctacaatgtgctttgcactatgtacacagatattatggacccaatgcttaacgcagtccaggagcagtttggcggcaatccctttgccactgccactactgataatgccaccaccaccaccagccaaccttcaaggatggagaattgtgaccctctccccaacccctggacttccacacatggaggctcaggtagcaggcaaggaaggcaggatggggatcaggatgcacctgacattagaaataggtttccaaactttctgggtattataaggctctatgactatctccagcaattacacgagaacccccagtccctaggaacttatctacaggggactgcatctgccctcagccaaagccaggaaccaccaccatcagtaaacagagttcccccatcgtcaccctcatctcaggagcctgggtcaggccagcctctccccgaggagtcagtagcaatcaagggaaggtcctcctgcccagctttcctgagataccccacagagaacagtactggacaaggtggagaccaagatggtgcagggaaaagctctactggacatagcacaaacttgcctgatcttgtctcggggctgggagattctgccaacagggttccatttgctcccttatctttttcccccacggcagccattcctggaatccctgagcctccctggctgccatccccggcttatccaagatctctgaggccagatggcatgaatccagctccacagttacaggatgagatacaaccacagctgccactgctgatgcaccttcaggcagccatggcaaacccccgtgccctgcaagccctgcggcagattgagcagggtctacaggtcctagctactgaagcacctcgcctcctactctggttcatgccttgcctagcagggacgggtagtgtggcaggaggtatagagtctagagaagatccccttatgtctgaggatcctctcccaaatccacctcctgaggtgttcccagcactggactctgcagagctgggcttcctttcccctccctttctccatatgctgcaagatttagttagtacaaatccccagcagctgcagcctgaggctcactttcaggtgcagctggagcaactgcggtccatgggctttctgaatcgtgaagccaatcttcaggccctcattgctacggggggcgacgtggatgctgctgtggagaagctgagacagtcgtaggagccttattcattcaaaccatacgttttcctctgtgcctttttcccatatcctagttccctagctctcccatttttgaatacagctgcattataaaccaaatttactatgaagtcctttgctgtggaggcaatgttgttccagagtcaacgaggaagactaatggccaaaacatagtggaggtgctgtgtgtgagtcaaccacttgtaccactataccactggggggccccagtctaagctctgcttatgcctatcttgagatgcaattacacccaatttccaatgtgaaaa

SEQ ID NO: 16 - protein, 655 aa, Homo sapiens
MAKGGEALPQGSPAPVQDPHLIKVTVKTPKDKEDFSVTDTCTIQQLKEEISQRFKAHPDQLVLIFAGKILKDPDSLAQCGVRDGLTVHLVIKRQHRAMGNECPAASVPTQGPSPGSLPQPSSIYPADGPPAFSLGLLTGLSRLGLAYRGFPDQPSSLMRQHVSVPEFVTQLIDDPFIPGLLSNTGLVRQLVLDNPHMQQLIQHNPEIGHILNNPEIMRQTLEFLRNPAMMQEMIRSQDRVLSNLESIPGGYNVLCTMYTDIMDPMLNAVQEQFGGNPFATATTDNATTTTSQPSRMENCDPLPNPWTSTHGGSGSRQGRQDGDQDAPDIRNRFPNFLGIIRLYDYLQQLHENPQSLGTYLQGTASALSQSQEPPPSVNRVPPSSPSSQEPGSGQPLPEESVAIKGRSSCPAFLRYPTENSTGQGGDQDGAGKSSTGHSTNLPDLVSGLGDSANRVPFAPLSFSPTAAIPGIPEPPWLPSPAYPRSLRPDGMNPAPQLQDEIQPQLPLLMHLQAAMANPRALQALRQIEQGLQVLATEAPRLLLWFMPCLAGTGSVAGGIESREDPLMSEDPLPNPPPEVFPALDSAELGFLSPPFLHMLQDLVSTNPQQLQPEAHFQVQLEQLRSMGFLNREANLQALIATGGDVDAAVEKLRQS

SEQ ID NO: 17 - DNA, 3545 bp, Homo sapiens
gggcggccgggtggcggcggcggcatggcggagccgagcggggccgagacgaggccccccattcgggtcaccctcaagacccccaaggacaaggaggaaattgtgatctgcgatcgagcctcggtcaaggagttcaaagagtcctgatcttcgcaggcaaaaggacgggctcactgtccagctgccactgcttcttccccgcttcacccgccacccctgcagtggaagccggaggagcagagtgctactgcgtccatactctgggctctgccaacttcatcctgagatgctgtcacagatgatctgatgcgtcacatgatcctgagatcagccacatgctcggaatccagccatgatgcagagagcatccctggagggtaatgttcagtgctgcccgggatccgacagctcatcctcccatggagcccctcgccccccactcggggaccagccaggtgcactggggtcagggatgttcaaaacccccagctgatgcagaacttgcccagaaccccgacttaacccccaactgcaggagcaaacccagagtcactctccatggaaatctcccggaggtttaaggctcagcaggatcagctggatcctcaaggatggggacacactgaaccagcacggaatctctggtcatcaagacccctcagaaggctcaagatccagctctccacacctgaccctgcctcagcaccctccaccacgcctccagccctccacctctggcagtgcctcttcagatgctggctggtggggggccctctccgggggctggggagggatcccccctctggctttgggggcatcctggggctgggcagcctaggcggagctgcagcagcagatgcagcggcagctgatgtccaatcatggagaaccccctggtccaggatatgatgtctaacccttatggccaacccccagatgcagcagttgatggagcggaaccaataaccctgaactcatgaggcagacaatggagcttgctagagatgatgcggaaccaggaccgggccctgagcaacctttaatgccctccgccgcatgtacacggacatccaggagcccacagtttggcaacaatcccttctcttccctggccgggaacgcctctgcggactgagaatcgagagcccctccctaaccccctcccaggcccccgggtccggtggggagggcaccggaggacccgacagtctcgaacccctttgggatcaatgcggctagctagcccagaaatgcaagccctcctccagcagatctctgagtgtgatctcagcaccctacatgcgcagcatgatgcagacgtgctgctcagatgatggtgaatgtgccgctcttcgcgggggctccgcctgcagctcccagtcttcctgcagcagatgcagccttaccaatccccgagccatgcaggcattgctgcagatccagcagggactacagaccttgcagaccgaggcccctgggctggtacccagccttggctcctttgggatatcccggaccccgcccccacttcctcaccagccagcagcaactcatgcagcacagacgccagaagtgagattaatcgtgaggctaacctgcagagagactgctgggctcccatccctcgatgtcagcattcgttccgtcttctccctcatccccccttagctctgtctgagacctggtctccttgagcagtgcttcttgccccacaccacttgaggaaccagctctctggtttcttatgtttacttttggtacccaaccaatgctagaattttccttctcggcccctattctagcaccctcagcaggcagcaacgcagggtctacgcccgagcacgccagccacatcttctccaacaggggcttccagcgccgatgatccagcttttggctggaagtggaaactcacaggtgtcagcagcagctggagcagctcaactccatgggcttcatcggccctgattgccacaggaggggacatcaacgcagctatcgctctcctaatccctcggcccatgcctcctgcctctccccgttcttctgtcaatccttaccctctgcagcttgtcctccctttccaaacagcagggtgactttagaggcatgggctccaaattatggttttactgctacgtctctaacagactcttctctctacttaaacagttttcacagtttcattgattgactctacttgcaatctttaaactttcagtggctgtgcagagtcgaggtactggaacatagtcttccatctataccactagggttttgactctcttccttctttttctccctaccccccaacccctagcttgctctgaaggaggagcaggtgaaacaggtggtaatttggttcattcagcactttttggtgggaactgttggaaattccagggtaaggaggaagatgcctgttctccctgtctgaagagggagatgagacagctctctggacaggaattaacaaacgctggagcagcccagaggaaattcgtgtgaaagaggagggaatgagattattcggaggaagggaatgggggagacagcctgagtaaaaggcttggaagttggaattagcagtggggagcagaagcactcatagctcttttaggcagaagaatccaggcccgagctggcagaagagacttagagatgctaatggaatttaaactgaaaaaaggagcccaatgaagctaagcgccacgccccacaaggggtcatattggctttagttcctcaagcatatgtgcttatatgcacacacacacacatttccatggacccaggtcttgcttgtgtccccaggcaccagtagtttgagcccccctcaaaagacatgaagggggttggggtctgtgtgagtagtggggaggtgcatgtgtatccacatgtgtgcatacactcttaagttgggtgggaagtggattccttgttggtttctggttcagagtgctctcccaccaccagagacaagtgggtaaaaaggtcagtccattgcaggaatatatatccgggagagctaggtcccttggggctctggatgctgggtaacccagaggtaatgtgggtaccctttctgaagctgtcagggctgtgactagcacccttatcacccctcactgccttgtgggaatagtagagggtttttttcctccagagcccctggcctttcagttcttaactatttccctccaggccagaaagttttctttgaggaaggagaggagagggtggcaatgatgcctttgatctggaattggacatttctctgtcagagcacagaggaggctcatatcacctcttccctctcctacttggcccagctgcttggaggaccgaccccatggctgagaatatgacggcaagaggaacagagtttgctccaagtgggaaagggtcccaagcagtccagagaagatgtctgtgtggctttccctccctgcctcccccagctcccacactggcctttgtaaataaatggcgtggtctttgttgtgaaaaaaaaaaaaaaaaaaaaaaaaaaaaaaaaaaaaaaa

SEQ ID NO: 18 - protein, 601 aa, Homo sapiens
MAEPSGAETRPPIRVTVKTPKDKEEIVICDRASVKEFKEEISRRFKAQQDQLVLIFAGKILKDGDTLNQHGIKDGLTVHLVIKTPQKAQDPAAATASSPSTPDPASAPSTTPASPATPAQPSTSGSASSDAGSGSRRSSGGGPSPGAGEGSPSATASILSGFGGILGLGSLGLGSANFMELQQQMQRQLMSNPEMLSQIMENPLVQDMMSNPDLMRHMIMANPQMQQLMERNPEISHMLNNPELMRQTMELARNPAMMQEMMRNQDRALSNLESIPGGYNALRRMYTDIQEPMFSAAREQFGNNPFSSLAGNSDSSSSQPLRTENREPLPNPWSPSPPTSQAPGSGGEGTGGSGTSQVHPTVSNPFGINAASLGSGMFNSPEMQALLQQISENPQLMQNVISAPYMRSMMQTLAQNPDFAAQMMVNVPLFAGNPQLQEQLRLQLPVFLQQMQNPESLSILTNPRAMQALLQIQQGLQTLQTEAPGLVPSLGSFGISRTPAPSAGSNAGSTPEAPTSSPATPATSSPTGASSAQQQLMQQMIQLLAGSGNSQVQTPEVRFQQQLEQLNSMGFINREANLQALIATGGDINAAIERLLGSQLS